SEQUENCE LISTING (63 sequences)

SEQ ID NO: 1 - protein, 109 aa, synthetic construct
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKR

SEQ ID NO: 2 - protein, 6 aa, synthetic construct
QSVDNN

SEQ ID NO: 3 - protein, 10 aa, synthetic construct [sequence omitted]

SEQ ID NO: 4 - protein, 11 aa, synthetic construct
RASQSVDNNLV

SEQ ID NO: 5 - protein, 7 aa, synthetic construct
GASTRAT

SEQ ID NO: 6 - protein, 120 aa, synthetic construct
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSA

SEQ ID NO: 7 - protein, 10 aa, synthetic construct
GGSISSFNYY

SEQ ID NO: 8 - protein, 7 aa, synthetic construct
IYYSGST

SEQ ID NO: 9 - protein, 11 aa, synthetic construct
ARGYNWNYFDY

SEQ ID NO: 10 - protein, 7 aa, synthetic construct
SFNYYWS

SEQ ID NO: 11 - protein, 16 aa, synthetic construct
YIYYSGSTYSNPSLKS

SEQ ID NO: 12 - protein, 9 aa, synthetic construct
GYNWNYFDY

SEQ ID NO: 13 - DNA, 327 bp, synthetic construct
  source 1..327: mol_type = other DNA
gagatcgtgatgacccagagtcccgccaccctttccgtaagccccggggagcgggctacactgtcatgccgagcgtcacagagcgtcgacaacaacctggtgtggtaccagcagaagcctggacaggccccgcgcctgctcatttacggcgcttctacccgtgctaccggcatcccagctcgtttttcaggctccggatccgggaccgagttcaccctgacgatctcttctttgcagagcgaagattttgcggtttattactgtcaacagtacaacaattggccgccgtggaccttcggtcagggcaccaaggtggagatcaagcgc

SEQ ID NO: 14 - DNA, 360 bp, synthetic construct
  source 1..360: mol_type = other DNA
caggtgcagctgcaggagagcggccctggcctcgtgaagccctctcagactctttcgctaacctgcactgtctcgggcgggtccatcagctccttcaactactactggagttggatccgccaccaccccggcaaaggcctggagtggattggctacatctactactctggttccacctactccaatccatctctgaaatcccgcgtgactatttctgtcgatacctccaagaaccagttctccctgaccctgagttccgtgactgctgcggacaccgccgtgtactactgtgcccgcggctacaactggaactatttcgactattggggccagggaactctcgtcaccgtgtcgagcgca

SEQ ID NO: 15 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPITFGQGTRLEIKR

SEQ ID NO: 16 - protein, 11 aa, synthetic construct
QSLLHSNGYNY

SEQ ID NO: 17 - protein, 9 aa, synthetic construct
MQGLQTPIT

SEQ ID NO: 18 - protein, 16 aa, synthetic construct
RSSQSLLHSNGYNYLD

SEQ ID NO: 19 - protein, 7 aa, synthetic construct
LGSNRAS

SEQ ID NO: 20 - protein, 126 aa, synthetic construct
QLVESGGGVVQPGRSLRLSCAASGFAFSSYGMHWVRQAPGKGLEWVAVISYDGNNKYYADSVKGRFTISRDNSKNTLYLQMNSLRAEDTAVYYCARDLVVRGIRGYYYYFGMDVWGQGTTVTVSSA

SEQ ID NO: 21 - protein, 8 aa, synthetic construct
GFAFSSYG

SEQ ID NO: 22 - protein, 8 aa, synthetic construct
ISYDGNNK

SEQ ID NO: 23 - protein, 20 aa, synthetic construct
ARDLVVRGIRGYYYYFGMDV

SEQ ID NO: 24 - protein, 5 aa, synthetic construct
SYGMH

SEQ ID NO: 25 - protein, 17 aa, synthetic construct
VISYDGNNKYYADSVKG

SEQ ID NO: 26 - protein, 18 aa, synthetic construct
DLVVRGIRGYYYYFGMDV

SEQ ID NO: 27 - DNA, 339 bp, synthetic construct
  source 1..339: mol_type = other DNA
gacatcgtgatgacccagagccccctgagcctgccagtgactccgggggagcccgcctccatctcatgccgatcttctcagagtctactccactcaaatggctacaactacttggattggtacttgcagaagcctggacagagccctcagctgctgatttacctgggcagcaaccgcgcttctggtgtccccgacaggttttcgggctccggcagtggcaccgactttaccctgaagatatctcgggtggaggccgaggacgtgggcgtctactactgtatgcaagggctgcagactcccatcacgttcggtcagggtactcgcctggagatcaaacgc

SEQ ID NO: 28 - DNA, 378 bp, synthetic construct
  source 1..378: mol_type = other DNA
cagctggtggagagcggtgggggtgtggtgcagccagggcgttctctgcgcctttcctgtgctgcttccggcttcgcgttctcgagctatggtatgcattgggtgcgccaggccccgggcaaaggcctggagtgggtcgccgtgattagctacgacggcaacaacaagtactacgcggactccgtgaagggccgcttcaccatctcccgcgacaactccaagaacacgctttacctccagatgaattccctcagagcagaagacaccgcggtctactattgcgcccgggatctggtagttcgtggcatccgcggctattactactatttcggaatggatgtgtggggccagggcacaaccgtcaccgtgtcgtctgct

SEQ ID NO: 29 - protein, 107 aa, synthetic construct
DIQLTQSPSSLSASVGDRVTITCRASQGIRNDLGWYQQKPGKAPKRLIYAASSLQNGVPSRFSGSGSGTEFTLTISSLQPEDFATYYCLQHNTYPAFCQGTKVEIKR

SEQ ID NO: 30 - protein, 6 aa, synthetic construct
QGIRND

SEQ ID NO: 31 - protein, 8 aa, synthetic construct
LQHNTYPA

SEQ ID NO: 32 - protein, 11 aa, synthetic construct
RASQGIRNDLG

SEQ ID NO: 33 - protein, 7 aa, synthetic construct
AASSLQN

SEQ ID NO: 34 - protein, 132 aa, synthetic construct
QVQLEQSGPGLVKPSQNLSLTCTVSGGSISSGGYFWSWIRQHPGKGLEWIGYIYYSGNTYYNPSLKSRVTISVDTSKNQFSLKLSSVTAADTAVYYCARDYYYDTSGFSYRYDWYYGMDVWGQGTTVTVSSA

SEQ ID NO: 35 - protein, 10 aa, synthetic construct
GGSISSGGYF

SEQ ID NO: 36 - protein, 7 aa, synthetic construct
IYYSGNT

SEQ ID NO: 37 - protein, 23 aa, synthetic construct
ARDYYYDTSGFSYRYDWYYGMDV

SEQ ID NO: 38 - protein, 7 aa, synthetic construct
SGGYFWS

SEQ ID NO: 39 - protein, 16 aa, synthetic construct
YIYYSGNTYYNPSLKS

SEQ ID NO: 40 - protein, 21 aa, synthetic construct
DYYYDTSGFSYRYDWYYGMDV

SEQ ID NO: 41 - DNA, 321 bp, synthetic construct
  source 1..321: mol_type = other DNA
gacatccagcttacccagagcccatcgtctttgtccgcttccgtaggggacagggtcaccatcacctgtcgtgccagccaaggcatccgcaacgacctgggctggtaccagcagaagcctggcaaggcccccaagcgcctgatttacgcggcttcttctcttcagaatggggtgccttccagattttcaggctccggttctggcaccgagttcaccctcactatctcgtccttgcagccggaggactttgccacctattactgcctgcagcacaacacgtacccagcgttctgtcagggcacgaaggtggagatcaagcgt

SEQ ID NO: 42 - DNA, 396 bp, synthetic construct
  source 1..396: mol_type = other DNA
caggtgcagctggaacagagcggacccgggctggtgaagcccagccagaacctgtctctgacctgcactgtgtcgggcggctccatctctagtgggggctatttctggagttggattcgccagcatcccggcaaaggcctagagtggattggatacatctattactccggcaacacttactacaacccgtcactcaaatcccgcgtcacaatctccgtcgatacctccaagaaccagttcagcctgaagctgagctccgttactgctgcggacaccgccgtgtactactgcgcccgggactattactacgacaccagcggcttcagctaccgctacgattggtactacggaatggatgtgtggggccagggtacgaccgtcaccgtgtcctctgca

SEQ ID NO: 43 - protein, 114 aa, synthetic construct
LDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTHPTFGQGTKVEIKR

SEQ ID NO: 44 - protein, 9 aa, synthetic construct
MQALQTHPT

SEQ ID NO: 45 - protein, 111 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGPNTWGQGTLVTV

SEQ ID NO: 46 - protein, 8 aa, synthetic construct
GGTFSSYA

SEQ ID NO: 47 - protein, 8 aa, synthetic construct
IIPIFGTA

SEQ ID NO: 48 - protein, 6 aa, synthetic construct
ARGPNT

SEQ ID NO: 49 - protein, 5 aa, synthetic construct
SYAIS

SEQ ID NO: 50 - protein, 17 aa, synthetic construct
GIIPIFGTANYAQKFQG

SEQ ID NO: 51 - protein, 4 aa, synthetic construct
GPNT

SEQ ID NO: 52 - DNA, 342 bp, synthetic construct
  source 1..342: mol_type = other DNA
cttgatgtggtgatgacccagagcccactgagccttccggtgactccgggggagccagcctccatctcttgccgatcctctcaaagcctgctgcattcaaatggctacaactatttggattggtacctgcagaagccgggacagtcccctcagctcctcatctacctgggcagtaaccgcgcctccggtgtccccgaccgcttctcgggctccggttctggcaccgacttcaccctgaagatttctcgggtggaggctgaagacgtgggcgtgtactactgcatgcaggccttgcagacacaccccacatttggtcaggggaccaaggtggagatcaagcgc

SEQ ID NO: 53 - DNA, 333 bp, synthetic construct
  source 1..333: mol_type = other DNA
caggtccagctggtgcagagcggggccgaggtgaagaagcctggctcctccgttaaagtgtcctgtaaggcttccggtggcacgttctccagctacgcgattagttgggtccgccaggctcccggacagggtctggagtggatgggcggcatcatccccatcttcggaacggccaactacgcgcagaaatttcagggcagggtcaccatcaccgcggacgagagcacttcgaccgcctacatggagctgtcttctctgcgctcggaggacaccgcagtttattactgtgctcgtggccctaacacctggggccagggcactctagtaaccgtg

SEQ ID NO: 54 - protein, 15 aa, synthetic construct
GGGGSGGGGSGGGGS

SEQ ID NO: 55 - protein, 120 aa, synthetic construct
QLVESGGGVVQPGRSLRLSCAASGFAFSSYGMHWVRQAPGKGLEWVAVISYDGNNKYYADSVKGRFTISRDNSKNTLYLQMNSLRAEDTAVYYCARDLVVRGIRGYYYYFGMDVWGQGTT

SEQ ID NO: 56 - protein, 6 aa, synthetic construct
VTVSSA

SEQ ID NO: 57 - protein, 10 aa, synthetic construct
EQKLISEEDL

SEQ ID NO: 58 - protein, 6 aa, synthetic construct
AAATTT

SEQ ID NO: 59 - protein, 42 aa, synthetic construct
PAPRPPTPAPTIASQPLSLRPEACRPAAGGAVHTRGLDFACD

SEQ ID NO: 60 - protein, 24 aa, synthetic construct
IYIWAPLAGTCGVLLLSLVITLYC

SEQ ID NO: 61 - protein, 42 aa, synthetic construct
KRGRKKLLYIFKQPFMRPVQTTQEEDGCSCRFPEEEEGGCEL

SEQ ID NO: 62 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 21 aa, synthetic construct
MALPVTALLLPLALLLHAARP

(19) United States
(12) Patent Application Publication
(10) Pub. No.: US 2026/0265406 A1
(43) Pub. Date: Sep. 10, 2026

(54) Anti-GPNMB Chimeric Antigen Receptors and Methods of Use

(71) Applicant: UTI Limited Partnership, Calgary (CA)

(72) Inventors: Douglas J. Mahoney, Calgary (CA); Franz J. Zemp, Calgary (CA)

(21) Appl. No.: 19/127,386

(22) PCT Filed: Nov. 7, 2023
(86) PCT No.: PCT/IB2023/061240

§ 371 Date: May 5, 2025

Related U.S. Application Data
(60) Provisional application No. 63/423,405, filed on Nov. 7, 2022.

(51) Int. Cl. C07K 16/30; A61K 40/11; A61K 40/31; A61K 40/42; A61P 35/00; C12N 5/0783

(52) U.S. Cl. CPC C07K 16/30; A61K 40/11; A61K 40/31; A61K 40/4202; A61P 35/00; C12N 5/0636; C07K 2317/53; C07K 2317/622; C07K 2319/02; C07K 2319/03; C12N 2510/00

(57) ABSTRACT

Provided are glycoprotein NMB (GPNMB)-binding chimeric antigen receptors (CARs). In some embodiments, a CAR of the present disclosure comprises an extracellular GPNMB-binding domain (e.g., a single chain antibody, such as an scFv), a transmembrane domain, and one or more intracellular signaling domains. Nucleic acids and expression constructs encoding such CARs are also provided. Also provided are cells (e.g., immune cells, such as T cells) comprising the nucleic acids and expression constructs. Aspects of the present disclosure further include compositions comprising a population of such cells expressing the GPNMB-binding CAR on their surface, as well as methods of administering such compositions to treat a condition associated with GPNMB expression and/or activity in a subject in need thereof. Non-limiting examples of conditions associated with GPNMB expression and/or activity include cancer (e.g., sarcomas, such as soft tissue sarcomas), neurodegenerative diseases, and the like.

### CROSS-REFERENCE TO RELATED APPLICATIONS

This application claims the benefit of U.S. Provisional Patent Application No. 63/423,405, filed Nov. 7, 2022, which application is incorporated herein by reference in its entirety.

### INCORPORATION BY REFERENCE OF SEQUENCE LISTING XML

A sequence listing is provided herewith as a Sequence Listing XML, “UTIP-001WO_SEQ_LIST”, created on Nov. 6, 2023 and having a size of 59,559 bytes. The contents of the Sequence Listing XML are incorporated by reference herein in their entirety.

### INTRODUCTION

Glycoprotein NMB (GPNMB) is a glycoprotein that consists of a large extracellular domain (ECD) and a short 53 amino acid cytoplasmic tail, connected by a single pass transmembrane domain. The ECD contains an N-terminal signal peptide, an RGD motif that binds to integrin receptor subunits, several N-glycosylation sites, a proline-rich region and an Ig-like polycystic kidney disease (PKD) domain. The ECD can be shed from the plasma membrane by ADAM10, which can then act as a paracrine factor to activate multiple signaling pathways in a variety of cells. The intracellular domain harbors a half immunoreceptor tyrosine-based activation motif (hemITAM) and a dileucine motif, the latter of which functions as an endosomal/lysosomal sorting motif in QNR-71. Functionally relevant domains within GPNMB include the extracellular RGD motif, which is critical for engagement of integrin receptors and promoting cell adhesion. In addition, the cytoplasmic hemITAM domain can be phosphorylated at tyrosine residue 525 following GPNMB engagement, which leads to the activation of several downstream signaling pathways.

GPNMB is highly expressed in a wide array of tumors including glioblastoma and astrocytoma, breast cancer, hepatocellular carcinoma, gastric adenocarcinoma, uveal melanoma, lung cancer, malignant cutaneous melanoma, and sarcomas. Since its discovery, both in vitro and in vivo studies have revealed GPNMB to be a key promoter of tumor growth and an enhancer of malignant phenotypes, such as tumor invasion and metastasis, in prostate, breast, lung and pancreatic cancers.

### SUMMARY

Provided are glycoprotein NMB (GPNMB)-binding chimeric antigen receptors (CARs). In some embodiments, a CAR of the present disclosure comprises an extracellular GPNMB-binding domain (e.g., a single chain antibody, such as an scFv), a transmembrane domain, and one or more intracellular signaling domains. Nucleic acids and expression constructs encoding such CARs are also provided. Also provided are cells (e.g., immune cells) comprising the nucleic acids and expression constructs. Aspects of the present disclosure further include compositions comprising a population of such cells expressing the GPNMB-binding CAR on their surface, as well as methods of administering such compositions to treat a condition associated with GPNMB expression and/or activity in a subject in need thereof. Non-limiting examples of conditions associated with GPNMB expression and/or activity include cancer, neurodegenerative diseases, tissue-remodeling, and the like.

### BRIEF DESCRIPTION OF THE FIGURES

FIG. 1: Genomics assessment of cell-surface genes on alveolar soft part sarcoma (ASPS). Bioinformatics pipeline to identify potentially overexpressed cell-surface proteins in ASPS vs. Normal tissue from microarray data in GSE13433 (a). Expression of 11 candidate cell-surface ASPS genes derived from the pipeline across three published ASPS microarray datasets (b). GPNMB expression across different pediatric and AYA tumors from two separate publicly available datasets. Dashed line=median house-keeping gene expression; Dotted line=one standard deviation of the mean (c). Surgical time course of an ASPS patient (d; not represented is initial stereotactic radiosurgery for right parietal lobe lesion present at diagnosis, 02-2016). Each of these resections were stained for GPNMB (e), with each roman numeral corresponding to a surgery listed above.

FIG. 2: GPNMB and TFE staining of three primary ASPS tumor resections stained for GPNMB and TFE3. Primary 1 was resection from a primary tumor located in the upper thigh from a 14yo female. Primary 2 was a resection from a primary tumor located in the upper right shoulder of a male. Primary 3 was a resection from a primary tumor located in the thigh of a 30yo female.

FIG. 3: Normal Proteomics DB for GPNMB. Human Protein Map and Wang et al. were used to assess the level of GPNMB (circle) expression across tissues in non-malignant samples. In addition, CD276 (triangle) and HER2 (cross) were included as established CAR targets and 14 housekeeping genes (https://www.genomics-online.com/resources/16/5049/housekeeping-genes/): ACTB, B2M, GAPDH, GUSB, HMBS, HPRT1, PGK1, PPIA, RPL13A, RPLP0, SDHA, TBP, TFRC, and YWHAZ. Since the range of the mass spectrometry (MS) expression varies and can be extreme, the MS values were capped at the 90th percentile across 17 proteins within the dataset. Then, the MS values were scaled from [minimum, maximum] expression to [0, 1].

FIG. 4: Normal Tissue Histology Scores≥2. Represented images of tissues that stained ≥2 on a 0-3 scale including heart (a), tonsil (b), lymph node (c), skin (d), and placenta (e).

FIG. 5: Adverse event profiles between CDX-011 and SGN-035 trials. Graphed results of appendix II. All adverse events (a) and serious adverse events (≥3; b). 483 patients from 6 CDX-011 trials (NCT00412828, NCT00704158, NCT01156753, NCT01997333, NCT02363283, NCT02302339). 356 patients from 6 SGN-035 trials (NCT01100502, NCT00848926, NCT01807598, NCT01716806, NCT01352520, NCT01461538). All trials were evaluated with NCI-CTCAE (v3 of v4) adverse event criteria.

FIG. 6: GPNMB CAR and targeting ASPS patient-derived cell line. Schematic of the GPNMB41bbζ (GPNMB) CAR construct (a). Immunofluorescent staining of a patient-derived ASPS cell line for GPNMB, TFE3 and DAPI (b). Flow cytometry for cell surface expression of GPNMB on a patient-derived ASPS cell line for GPNMB (c). GPNMB (left) and TFE3 (right) staining of ASPS cell line orthotopically implanted intramuscularly in NSG-MHC^{KO} mice 35 days post tumor implantation (d). Representative flow cytometric analysis (left) of GPNMB CAR or an empty-CAR vector into healthy donor 1 T cells with corresponding transduction efficiency (middle; 5 MOI) in healthy donor 1 (D1) and donor 2 (D2) T cells. Transduction efficiency as measured by EGFP of CD4+ and CD8+ cells (right; e). Microscopy of 24-hour co-culture between patient-derived ASPS cell line (red) and CAR T-cells (green) and quantification of cytotoxic activity as measured by luminescent assay at listed ratios of CAR T to ASPS target cells (f). IFNγ and IL-2 secretion of CAR T-cells at 1:1 ratio in media measured 24 hours after co-culture. Solid bars are D1, hashed lines are D2 (g).

FIG. 7: Testing different GPNMB-binding scFvs in a preclinical CAR construct. Using identical CAR and plasmid backbones, four different GPNMB-binding scFvs were tested. Primary human T cells were transduced at 10 MOI and expanded for 8 days following transduction. Transduction efficiency was measured by flow cytometry and reported as percent GFP positive (a). CAR surface expression was measured by flow cytometry using an anti-MYC antibody and MFI reported (b). Represented figures of GPNMB CAR T-cells (GFP—green) and ASPS patient-derived xenograft (PDX)-derived cell line (red—mCherry) after 20 hours of co-culture (c). Quantification of cytotoxic activity as measured by luminescent assay at listed rations of CAR T to ASPS target cells (d) and IFNγ and IL-2 secretion of CAR T-cells at 1:1 ratio in media measured 24 hours after co-culture. All data was from the same healthy donor, with freshly harvested, activated, and transduced T cells performed three independent times.

FIG. 8: GPNMB CAR in ASPS xenograft in vivo primary tumor. Luminescent images of animals bearing 42-day ASPS^{mcherry/FLUC} xenografts (Day 0), and treated with 1e5, 1e6, or 5e6 GPNMB CAR T-cells, 5e6 CD19 CAR T-cells from healthy donor 1, or untreated (control) and imaged on indicated days (a; 1e4 CAR/mouse not shown). Kaplan-Meier curve of animals from day of tumor-initiation to treatment on day 42 (arrow) and followed for survival. Death was result of tumors reaching 17 mm in any direction (b). Expansion of CAR T-cells in blood following CAR T treatment (c). Flow cytometry of CAR expression via MYC tag on the surface of expanded CAR T-cells in the blood following 5e6 GPNMB CAR treatment. Representative histogram (left) and quantitated MFI flow values (right). Black line is representing MYC expression, grey line is EGFP expression, and dotted line corresponds to pre-implantation MYC tag expression (d). Effector (Teff), Effector memory (Tem) and Central Memory (Tcm) cell percentage in the blood of mice following 5e6 GPNMB CAR administration (e). Intravital microscopy of primary tumor ASPS^{mcherry/FLUC} model treated with 1e6 GPNMB CAR T-cells. ASPS xenograft (red), CD31 (vasculature; blue) and GPNMB CAR T-cells (GFP; green) are colors on stitched images of multiple Z-stacked images (~100 uM depth; f).

FIG. 9: Donor 2-CAR Intramuscular Tumors. Luminescent images of animals bearing 42-day ASPSmc/FLUC xenografts (Day 0), and treated with 1e5, 1e6, or 5e6 GPNMB CAR, 5e6 CD19 CAR from healthy donor 1, or untreated and imaged on indicated days (a). CAR T cells in blood as measured by flow cytometry following CAR T treatment (b).

FIG. 10: Systemic GPNMB CAR effectively targets ASPS CNS metastasis. Luminescent images of animals bearing 42-day ASPS^{mcherry/FLUC} xenografts (Day 0), and treated with 1e4, 1e5, or 1e6 GPNMB-CAR T-cells, 1e6 CD19 CAR T-cells, or untreated and imaged on indicated days. Red arrow depicts areas of putative spinal metastasis (a). Kaplan-Meier curve of animals from day of tumor-initiation to treatment on day 42 and followed for survival. (b). CAR T cells in blood as measured by flow cytometry following CAR T treatment (c). Weights of animals taken following CAR T administration and presented as a percent weight at treatment Day 0 (d). Data is a combined result of two experiments performed in healthy donor cells harvested, transduced, and expanded independently for control and 1e6 GPNMB and CD19 CAR T-cells (n=8). And one experiment (n=4) for 1e5 GPNMB CAR T-cells (b-d).

FIG. 11: Testing GPNMB CAR in ASPS patient T cells. ASPS patient had blood drawn during period of disease quiescence and patient T cells were transduced with the GPNMB CAR construct and CAR expression measured by flow cytometry (a). Microscopy of 24-hour co-culture between patient-derived ASPS cell line (red) and the same patient's CAR T-cells (green; left) and quantification of cytotoxic activity as measured by luminescent assay at listed ratios of CAR T to ASPS target cells (b; Diamonds=untransduced cells; Circles=CD19 CAR T; Square=GPNMB CAR). IFNγ and IL-2 secretion from CAR T-cells (Circle=CD19; Square=GPNMB) at 1:1 ratio, measured in in media 24 hours after co-culture (c). Luminescent images of animals bearing 42-day ASPS^{mcherry/FLUC} xenografts (Day 0), and treated with 1e6 GPNMB CAR T-cells or 1e6 CD19 CAR T-cells, or untreated and imaged on indicated days (d) and CAR T cells in blood as measured by flow cytometry following CAR T treatment (e).

FIG. 12: Comparison of Preclinical and Clinical GPNMB CAR Vector in Patient T Cells. Testing of Preclinical versus Clinical GPNMB CAR T construct in vitro on ASPS PDX-derived cell line. Quantification of cytotoxic activity as measured by luminescent assay at listed ratios of CAR T to ASPS target cells (a). IFNγ and IL-2 secretion of CAR T-cells at 1:1 ratio in media measured 24 hours after co-culture (b).

FIG. 13: CliniMACs validation runs were performed on two separate healthy donor apheresis products (HD1, HD2) or apheresis product from an epithelioid hemangioendothelioma (EHE) patient. Runs on the CliniMACs utilized listed volumes of lentiviral product produced under principles of GMP and qualified for clinical use. Total cells (a), viability and CAR % (b) of each product with a 12-day manufacturing product. Potency of each product was measured by co-culture cytotoxicity assays with ASPS PDX-derived cells (c) and subsequent cytokine secretion (d). Luminescent images of animals bearing 42-day ASPS^{mc/FLUC} intramuscular xenografts (Day 0) and treated with 5e6 or 5e5 GPNMB CAR T derived from HD1 validation run (e). Number of HD1 CARs in blood following CAR T treatment (f). Controls are bearing a subcutaneous tumor that lacks GPNMB expression.

FIG. 14: Representative images of GPNMB staining of TSC/mTOR-altered renal cell carcinoma (RCC) subtypes low-grade oncocytic tumor (LOT), eosinophilic solid and cystic (ESC) and eosinophilic vacuolated tumor (EVT), translocation-associated RCC (tRCC), and clear-cell RCC (a) and histological scoring of slides scored by a renal cancer pathologist (0-3 intensity X % Tumor positive=H-Index; b). Scale bar=50 μM. Panel of tRCC-associated cells lines (FU-UR1, S-TFE3, UOK-109, UOK-120, UOK-124, UOK-145, UOK-146) and ccRCC cell line (786-O, A498, ACHN) tested for cell surface GPNMB expression by flow cytometry. Translocation-associated RCC cell lines (GPNMB+) and a ccRCC cell line (GPNMB−) were subcutaneously implanted at treated with 5e6 GPNMB CAR from the HD1 clinical run described in FIG. 5 (d). Expansion of the HD1 product in these models (e) compared to the GPNMB−, non-responding tumor. ASPS PDX included as a positive control.

FIG. 15: 49 triple-negative breast cancer (TNBC) patients were scored by a certified breast cancer pathologist (0-3 intensity X % Tumor positive=H-Index). Representative images of High (150+), medium (40-149), and low (<50) scoring GPNMB from cores from separate patients (left) and scoring of the entire TMA (right;). Scale bar=50 μM. TNBC cell lines MDA-MB-231 and Hs578T cell surface expression of GPNMB compared to positive control ASPS (b). These lines were tested head-head for cytotoxicity and cytokine secretion when treated in co-culture experiment with HD1 clinical-grade GPNMB CAR (c). Luciferase-expressing Hs578T cells were implanted in the mammary fat pad of NSG-KO mice and treated with 1e6 GPNMB CAR-T from HD1 (d).

### DETAILED DESCRIPTION

Before the CARs, compositions, and methods of the present disclosure are described in greater detail, it is to be understood that the CARs, compositions, and methods are not limited to particular embodiments described, as such may, of course, vary. It is also to be understood that the terminology used herein is for the purpose of describing particular embodiments only, and is not intended to be limiting, since the scope of the CARs, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the CARs, compositions, and methods. The upper and lower limits of these smaller ranges may independently be included in the smaller ranges and are also encompassed within the CARs, compositions, and methods, subject to any specifically excluded limit in the stated range. Where the stated range includes one or both of the limits, ranges excluding either or both of those included limits are also included in the CARs, compositions, and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the CARs, compositions, and methods belong. Although any CARs, compositions, and methods similar or equivalent to those described herein can also be used in the practice or testing of the CARs, compositions, and methods, representative illustrative CARs, compositions, and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present CARs, compositions, and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the CARs, compositions, and methods, which are, for clarity, described in the context of separate embodiments, may also be provided in combination in a single embodiment. Conversely, various features of the CARs, compositions, and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present CARs, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Anti-GPNMB Chimeric Antigen Receptors

Aspects of the present disclosure include anti-glycoprotein NMB (GPNMB) chimeric antigen receptors (CARs). CARs are bioengineered molecules typically comprising an antigen-binding (targeting) domain fused to transmembrane and intracellular signaling domains of the T cell receptor complex. CARs function by redirecting the cytotoxic activity of T cells toward tumors bearing a predetermined surface expressed antigen. Historically, the effectiveness of CAR T therapy in solid tumors has been poor. Broad utilization of CAR T therapy for cancers such as soft tissue sarcomas (STS) faces several hurdles, including target selection. Targets for solid tumors like sarcomas have two main requirements: ubiquitous, high level, and stable cell surface expression and limited off-tumor targeting potential.

The CARs, CAR-T cells and methods of the present disclosure are based in part on the inventors' identification of GPNMB as a candidate CAR target by virtue of its high cell surface expression on cancers such as MITF-family fusion positive cancers such as alveolar soft tissue sarcoma (ASPS) and some renal cell carcinomas (RCC). However, the inventors determined that only CARs having an unpredictable subset of GPNMB-targeting binding domains (e.g., scFvs “G1” and “G2” described herein) exhibit robust cell surface expression/localization. Also unpredictably, among the CARs with robust cell surface localization, there was varying in vitro cytotoxic and cytokine secretion activity. CAR T-cells utilizing the GPNMB-targeting binding domain with highest cell surface expression and in vitro activity exhibited clear in vivo efficacy in models of primary disease and metastasis, with significant CAR T expansion and complete, durable tumor elimination. Also unpredictably, despite low cell surface expression of an anti-GPNMB CAR, some in vitro efficacy was also observed (e.g., “G4”). Moreover, CAR T-cells expressing CARs comprising the subset of GPNMB-targeting binding domains exhibiting robust cell surface expression/localization exhibit efficacy at unexpectedly low doses, e.g., 1e5 in an orthotopic model of ASPS primary and metastatic disease. Details of the CARs of the present disclosure will now be described.

A CAR of the present disclosure comprises an extracellular GPNMB-binding domain, a transmembrane domain, and one or more intracellular signaling domains. In certain embodiments, the extracellular GPNMB-binding domain comprises a single-chain antibody (e.g., an scFv) that specifically binds GPNMB.

The term “antibody” may include an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In some embodiments, the antibody is selected from an IgG, Fv, single chain antibody, scFv, Fab, F(ab′)2, F(ab′) or Fab′.

An immunoglobulin light or heavy chain variable region is composed of a “framework” region (FR) interrupted by three hypervariable regions, also called “complementarity determining regions” or “CDRs”. The extent of the framework region and CDRs can be defined based on databases known in the art. See, for example, “Sequences of Proteins of Immunological Interest,” E. Kabat et al., Sequences of proteins of immunological interest, 4th ed. U.S. Dept. Health and Human Services, Public Health Services, Bethesda, MD (1987), Lefranc et al. IMGT, the international ImMunoGeneTics information System®. Nucl. Acids Res., 2005, 33:D593-D597 (www.imgt.org/textes/IMGTScientificChart/), and/or V Base at vbase.mrc-cpe.cam.ac.uk/). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

In certain embodiments, the extracellular GPNMB-binding domain comprises an antibody that specifically binds GPNMB. The phrases “specifically binds”, “specific for”, “immunoreactive” and “immunoreactivity”, and “antigen binding specificity”, when referring to an antibody, refer to a binding reaction with an antigen which is highly preferential to the antigen or a fragment thereof, so as to be determinative of the presence of the antigen in the presence of a heterogeneous population of antigens (e.g., proteins and other biologics, e.g., in a sample). Thus, under designated immunoassay conditions, the specified antibodies bind to a GPNMB antigen (e.g., human GPNMB—UniProt Q96F58) and do not bind in a significant amount to other antigens present in the sample. Specific binding to an antigen under such conditions may require an antibody that is selected for its specificity for a particular antigen. For example, an anti-GPNMB antibody can specifically bind to GPNMB, and not exhibit comparable binding (e.g., does not exhibit detectable binding) to other proteins present in a sample.

In some embodiments, an antibody of a CAR of the present disclosure “specifically binds” GPNMB (e.g., human GPNMB) if it binds to or associates with the GPNMB with an affinity or Kₐ (that is, an equilibrium association constant of a particular binding interaction with units of 1/M) of, for example, greater than or equal to about 10⁵ M⁻¹. In certain embodiments, the antibody binds to GPNMB with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to GPNMB with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for GPNMB can be readily determined using conventional techniques, e.g., by biolayer interferometry (BLI); competitive ELISA (enzyme-linked immunosorbent assay); equilibrium dialysis; surface plasmon resonance (SPR) technology (e.g., the BIAcore 2000 instrument, using general procedures outlined by the manufacturer); by radioimmunoassay; and/or the like.

According to some embodiments, the extracellular GPNMB-binding domain comprises a single chain antibody that specifically binds GPNMB. In some instances, the single chain antibody is a single chain variable fragment (scFv). Single chain antibodies (e.g., scFvs) that may be employed include, but are not limited to, single chain antibodies comprising: the six CDRs of the variable light chain (V_{L}) polypeptide of antibody G1, G2 or G4 set forth in Table 1 below, where in some embodiments such single chain antibodies comprise a V_{L} polypeptide comprising an amino acid sequence having 70% or greater, 75% or greater, 80% or greater, 85% or greater, 90% or greater, 95% or greater, or 100% identity to the amino acid sequence of the V_{L} of antibody G1, G2 or G4 set forth in Table 1; and/or the six CDRs of the variable heavy chain (V_{H}) polypeptide of antibody G1, G2 or G4 set forth in Table 1 below, where in some embodiments such single chain antibodies comprise a V_{H} polypeptide comprising an amino acid sequence having 70% or greater, 75% or greater, 80% or greater, 85% or greater, 90% or greater, 95% or greater, or 100% identity to the amino acid sequence of the V_{H} of antibody G1, G2 or G4 set forth in Table 1. CDR numbering as per AbRSA program (Li et al. (2019) Protein Sci 28(8):1524-1531) accessed 2023.11.06.

TABLE 1
Amino Acid and Nucleotide Sequences of Example anti-GPNMB Antibody Variable
Regions

| ANTIBODY | REGION OF | | |
|---|---|---|---|
| NAME | ANTIBODY | AMINO ACID OR NUCLEOTIDE SEQUENCE | SEQ ID |
| G1 | scFv V_{L} Amino | EIVMTQSPATLSVSPGERATLSCRASQSVDNNLV | 1 |
| | Acid | WYQQKPGQAPRLLIYGASTRATGIPARFSGSGS | |
| | | GTEFTLTISSLQSEDFAVYYCQQYNNWPPWTFG | |
| | | QGTKVEIKR | |
| | scFv V_{L} CDR1 | QSVDNN | 2 |
| | (IMGT) | | |
| | scFv V_{L} CDR2 | GAS | N/A |
| | (IMGT) | | |
| | scFv V_{L} CDR3 | QQYNNWPPWT | 3 |
| | (IMGT) | | |
| | scFv V_{L} CDR1 | RASQSVDNNLV | 4 |
| | (Kabat) | | |
| | scFv V_{L} CDR2 | GASTRAT | 5 |
| | (Kabat) | | |
| | scFv V_{L} CDR3 | QQYNNWPPWT | 3 |
| | (Kabat) | | |
| | scFv V_{H} Amino | QVQLQESGPGLVKPSQTLSLTCTVSGGSISSFNY | 6 |
| | Acid | YWSWIRHHPGKGLEWIGYIYYSGSTYSNPSLKS | |
| | | RVTISVDTSKNQFSLTLSSVTAADTAVYYCARGY | |
| | | NWNYFDYWGQGTLVTVSSA | |
| | scFv V_{H} CDR1 | GGSISSFNYY | 7 |
| | (IMGT) | | |
| | scFv V_{H} CDR2 | IYYSGST | 8 |
| | (IMGT) | | |
| | scFv V_{H} CDR3 | ARGYNWNYFDY | 9 |
| | (IMGT) | | |
| | scFv V_{H} CDR1 | SFNYYWS | 10 |
| | (Kabat) | | |
| | scFv V_{H} CDR2 | YIYYSGSTYSNPSLKS | 11 |
| | (Kabat) | | |
| | scFv V_{H} CDR3 | GYNWNYFDY | 12 |
| | (Kabat) | | |
| | scFv V_{L} | GAGATCGTGATGACCCAGAGTCCCGCCACCCT | 13 |
| | Nucleotide | TTCCGTAAGCCCCGGGGAGCGGGCTACACTG | |
| | | TCATGCCGAGCGTCACAGAGCGTCGACAACAA | |
| | | CCTGGTGTGGTACCAGCAGAAGCCTGGACAG | |
| | | GCCCCGCGCCTGCTCATTTACGGCGCTTCTAC | |
| | | CCGTGCTACCGGCATCCCAGCTCGTTTTTCAG | |
| | | GCTCCGGATCCGGGACCGAGTTCACCCTGAC | |
| | | GATCTCTTCTTTGCAGAGCGAAGATTTTGCGGT | |
| | | TTATTACTGTCAACAGTACAACAATTGGCCGCC | |
| | | GTGGACCTTCGGTCAGGGCACCAAGGTGGAG | |
| | | ATCAAGCGC | |
| | scFv V_{H} | CAGGTGCAGCTGCAGGAGAGCGGCCCTGGCC | 14 |
| | Nucleotide | TCGTGAAGCCCTCTCAGACTCTTTCGCTAACCT | |
| | | GCACTGTCTCGGGGGGGTCCATCAGCTCCTTC | |
| | | AACTACTACTGGAGTTGGATCCGCCACCACCC | |
| | | CGGCAAAGGCCTGGAGTGGATTGGCTACATCT | |
| | | ACTACTCTGGTTCCACCTACTCCAATCCATCTC | |
| | | TGAAATCCCGCGTGACTATTTCTGTCGATACCT | |
| | | CCAAGAACCAGTTCTCCCTGACCCTGAGTTCC | |
| | | GTGACTGCTGCGGACACCGCCGTGTACTACTG | |
| | | TGCCCGCGGCTACAACTGGAACTATTTCGACT | |
| | | ATTGGGGCCAGGGAACTCTCGTCACCGTGTCG | |
| | | AGCGCA | |
| G2 | scFv V_{L} Amino | DIVMTQSPLSLPVTPGEPASISCRSSQSLLHSNG | 15 |
| | Acid | YNYLDWYLQKPGQSPQLLIYLGSNRASGVPDRF | |
| | | SGSGSGTDFTLKISRVEAEDVGVYYCMQGLQTPI | |
| | | TFGQGTRLEIKR | |
| | scFv V_{L} CDR1 | QSLLHSNGYNY | 16 |
| | (IMGT) | | |
| | scFv V_{L} CDR2 | LGS | N/A |
| | (IMGT) | | |
| | scFv V_{L} CDR3 | MQGLQTPIT | 17 |
| | (IMGT) | | |
| | scFv V_{L} CDR1 | RSSQSLLHSNGYNYLD | 18 |
| | (Kabat) | | |
| | scFv V_{L} CDR2 | LGSNRAS | 19 |
| | (Kabat) | | |
| | scFv V_{L} CDR3 | MQGLQTPIT | 17 |
| | (Kabat) | | |
| | scFv V_{H} Amino | QLVESGGGVVQPGRSLRLSCAASGFAFSSYGM | 20 |
| | Acid | HWVRQAPGKGLEWVAVISYDGNNKYYADSVKG | |
| | | RFTISRDNSKNTLYLQMNSLRAEDTAVYYCARDL | |
| | | VVRGIRGYYYYFGMDVWGQGTTVTVSSA | |
| | scFv V_{H} CDR1 | GFAFSSYG | 21 |
| | (IMGT) | | |
| | scFv V_{H} CDR2 | ISYDGNNK | 22 |
| | (IMGT) | | |
| | scFv V_{H} CDR3 | ARDLVVRGIRGYYYYFGMDV | 23 |
| | (IMGT) | | |
| | scFv V_{H} CDR1 | SYGMH | 24 |
| | (Kabat) | | |
| | scFv V_{H} CDR2 | VISYDGNNKYYADSVKG | 25 |
| | (Kabat) | | |
| | scFv V_{H} CDR3 | DLVVRGIRGYYYYFGMDV | 26 |
| | (Kabat) | | |
| | scFv V_{L} | GACATCGTGATGACCCAGAGCCCCCTGAGCCT | 27 |
| | Nucleotide | GCCAGTGACTCCGGGGGAGCCCGCCTCCATC | |
| | | TCATGCCGATCTTCTCAGAGTCTACTCCACTCA | |
| | | AATGGCTACAACTACTTGGATTGGTACTTGCAG | |
| | | AAGCCTGGACAGAGCCCTCAGCTGCTGATTTA | |
| | | CCTGGGCAGCAACCGCGCTTCTGGTGTCCCC | |
| | | GACAGGTTTTCGGGCTCCGGCAGTGGCACCG | |
| | | ACTTTACCCTGAAGATATCTCGGGTGGAGGCC | |
| | | GAGGACGTGGGCGTCTACTACTGTATGCAAGG | |
| | | GCTGCAGACTCCCATCACGTTCGGTCAGGGTA | |
| | | CTCGCCTGGAGATCAAACGC | |
| | scFv V_{H} | CAGCTGGTGGAGAGCGGTGGGGGTGTGGTGC | 28 |
| | Nucleotide | AGCCAGGGCGTTCTCTGCGCCTTTCCTGTGCT | |
| | | GCTTCCGGCTTCGCGTTCTCGAGCTATGGTAT | |
| | | GCATTGGGTGCGCCAGGCCCCGGGCAAAGGC | |
| | | CTGGAGTGGGTCGCCGTGATTAGCTACGACG | |
| | | GCAACAACAAGTACTACGCGGACTCCGTGAAG | |
| | | GGCCGCTTCACCATCTCCCGCGACAACTCCAA | |
| | | GAACACGCTTTACCTCCAGATGAATTCCCTCAG | |
| | | AGCAGAAGACACCGCGGTCTACTATTGCGCCC | |
| | | GGGATCTGGTAGTTCGTGGCATCCGCGGCTAT | |
| | | TACTACTATTTCGGAATGGATGTGTGGGGCCA | |
| | | GGGCACAACCGTCACCGTGTCGTCTGCT | |
| G3 | scFv V_{L} Amino | DIQLTQSPSSLSASVGDRVTITCRASQGIRNDLG | 29 |
| | Acid | WYQQKPGKAPKRLIYAASSLQNGVPSRFSGSGS | |
| | | GTEFTLTISSLQPEDFATYYCLQHNTYPAFCQGT | |
| | | KVEIKR | |
| | scFv V_{L} CDR1 | QGIRND | 30 |
| | (IMGT) | | |
| | scFv V_{L} CDR2 | AAS | N/A |
| | (IMGT) | | |
| | scFv V_{L} CDR3 | LQHNTYPA | 31 |
| | (IMGT) | | |
| | scFv V_{L} CDR1 | RASQGIRNDLG | 32 |
| | (Kabat) | | |
| | scFv V_{L} CDR2 | AASSLQN | 33 |
| | (Kabat) | | |
| | scFv V_{L} CDR3 | LQHNTYPA | 31 |
| | (Kabat) | | |
| | scFv V_{H} Amino | QVQLEQSGPGLVKPSQNLSLTCTVSGGSISSGG | 34 |
| | Acid | YFWSWIRQHPGKGLEWIGYIYYSGNTYYNPSLK | |
| | | SRVTISVDTSKNQFSLKLSSVTAADTAVYYCARD | |
| | | YYYDTSGFSYRYDWYYGMDVWGQGTTVTVSSA | |
| | scFv V_{H} CDR1 | GGSISSGGYF | 35 |
| | (IMGT) | | |
| | scFv V_{H} CDR2 | IYYSGNT | 36 |
| | (IMGT) | | |
| | scFv V_{H} CDR3 | ARDYYYDTSGFSYRYDWYYGMDV | 37 |
| | (IMGT) | | |
| | scFv V_{H} CDR1 | SGGYFWS | 38 |
| | (Kabat) | | |
| | scFv V_{H} CDR2 | YIYYSGNTYYNPSLKS | 39 |
| | (Kabat) | | |
| | scFv V_{H} CDR3 | DYYYDTSGFSYRYDWYYGMDV | 40 |
| | (Kabat) | | |
| | scFv V_{L} | GACATCCAGCTTACCCAGAGCCCATCGTCTTT | 41 |
| | Nucleotide | GTCCGCTTCCGTAGGGGACAGGGTCACCATCA | |
| | | CCTGTCGTGCCAGCCAAGGCATCCGCAACGAC | |
| | | CTGGGCTGGTACCAGCAGAAGCCTGGCAAGG | |
| | | CCCCCAAGCGCCTGATTTACGCGGCTTCTTCT | |
| | | CTTCAGAATGGGGTGCCTTCCAGATTTTCAGG | |
| | | CTCCGGTTCTGGCACCGAGTTCACCCTCACTA | |
| | | TCTCGTCCTTGCAGCCGGAGGACTTTGCCACC | |
| | | TATTACTGCCTGCAGCACAACACGTACCCAGC | |
| | | GTTCTGTCAGGGCACGAAGGTGGAGATCAAGC | |
| | | GT | |
| | scFv V_{H} | CAGGTGCAGCTGGAACAGAGCGGACCCGGGC | 42 |
| | Nucleotide | TGGTGAAGCCCAGCCAGAACCTGTCTCTGACC | |
| | | TGCACTGTGTCGGGGGGCTCCATCTCTAGTGG | |
| | | GGGCTATTTCTGGAGTTGGATTCGCCAGCATC | |
| | | CCGGCAAAGGCCTAGAGTGGATTGGATACATC | |
| | | TATTACTCCGGCAACACTTACTACAACCCGTCA | |
| | | CTCAAATCCCGCGTCACAATCTCCGTCGATAC | |
| | | CTCCAAGAACCAGTTCAGCCTGAAGCTGAGCT | |
| | | CCGTTACTGCTGCGGACACCGCCGTGTACTAC | |
| | | TGCGCCCGGGACTATTACTACGACACCAGCGG | |
| | | CTTCAGCTACCGCTACGATTGGTACTACGGAA | |
| | | TGGATGTGTGGGGCCAGGGTACGACCGTCAC | |
| | | CGTGTCCTCTGCA | |
| G4 | scFv V_{L} Amino | LDVVMTQSPLSLPVTPGEPASISCRSSQSLLHSN | 43 |
| | Acid | GYNYLDWYLQKPGQSPQLLIYLGSNRASGVPDR | |
| | | FSGSGSGTDFTLKISRVEAEDVGVYYCMQALQT | |
| | | HPTFGQGTKVEIKR | |
| | scFv V_{L} CDR1 | QSLLHSNGYNY | 16 |
| | (IMGT) | | |
| | scFv V_{L} CDR2 | LGS | N/A |
| | (IMGT) | | |
| | scFv V_{L} CDR3 | MQALQTHPT | 44 |
| | (IMGT) | | |
| | scFv V_{L} CDR1 | RSSQSLLHSNGYNYLD | 18 |
| | (Kabat) | | |
| | scFv V_{L} CDR2 | LGSNRAS | 19 |
| | (Kabat) | | |
| | scFv V_{L} CDR3 | MQALQTHPT | 44 |
| | (Kabat) | | |
| | scFv V_{H} Amino | QVQLVQSGAEVKKPGSSVKVSCKASGGTFSSYA | 45 |
| | Acid | ISWVRQAPGQGLEWMGGIIPIFGTANYAQKFQG | |
| | | RVTITADESTSTAYMELSSLRSEDTAVYYCARGP | |
| | | NTWGQGTLVTV | |
| | scFv V_{H} CDR1 | GGTFSSYA | 46 |
| | (IMGT) | | |
| | scFv V_{H} CDR2 | IIPIFGTA | 47 |
| | (IMGT) | | |
| | scFv V_{H} CDR3 | ARGPNT | 48 |
| | (IMGT) | | |
| | scFv V_{H} CDR1 | SYAIS | 49 |
| | (Kabat) | | |
| | scFv V_{H} CDR2 | GIIPIFGTANYAQKFQG | 50 |
| | (Kabat) | | |
| | scFv V_{H} CDR3 | GPNT | 51 |
| | (Kabat) | | |
| | scFv V_{L} | CTTGATGTGGTGATGACCCAGAGCCCACTGAG | 52 |
| | Nucleotide | CCTTCCGGTGACTCCGGGGGAGCCAGCCTCC | |
| | | ATCTCTTGCCGATCCTCTCAAAGCCTGCTGCAT | |
| | | TCAAATGGCTACAACTATTTGGATTGGTACCTG | |
| | | CAGAAGCCGGGACAGTCCCCTCAGCTCCTCAT | |
| | | CTACCTGGGCAGTAACCGCGCCTCCGGTGTCC | |
| | | CCGACCGCTTCTCGGGCTCCGGTTCTGGCACC | |
| | | GACTTCACCCTGAAGATTTCTCGGGTGGAGGC | |
| | | TGAAGACGTGGGCGTGTACTACTGCATGCAGG | |
| | | CCTTGCAGACACACCCCACATTTGGTCAGGGG | |
| | | ACCAAGGTGGAGATCAAGCGC | |
| | scFv V_{H} | CAGGTCCAGCTGGTGCAGAGGGGGGCCGAGG | 53 |
| | Nucleotide | TGAAGAAGCCTGGCTCCTCCGTTAAAGTGTCC | |
| | | TGTAAGGCTTCCGGTGGCACGTTCTCCAGCTA | |
| | | CGCGATTAGTTGGGTCCGCCAGGCTCCCGGA | |
| | | CAGGGTCTGGAGTGGATGGGGGGCATCATCC | |
| | | CCATCTTCGGAACGGCCAACTACGCGCAGAAA | |
| | | TTTCAGGGCAGGGTCACCATCACCGCGGACGA | |
| | | GAGCACTTCGACCGCCTACATGGAGCTGTCTT | |
| | | CTCTGCGCTCGGAGGACACCGCAGTTTATTAC | |
| | | TGTGCTCGTGGCCCTAACACCTGGGGCCAGG | |
| | | GCACTCTAGTAACCGTG | |

The present disclosure provides each of the polypeptides provided in Table 1, and each of the individual domains therein, as well as nucleic acids that encode such polypeptides and individual domains. Cells comprising such polypeptides and nucleic acids are also provided. As will be appreciated, the present disclosure also provides variants of any of the polypeptides and individual domains therein, where in some instances a variant polypeptide or domain thereof comprises an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to the parental/reference sequence, or a functional fragment thereof, where the variant retains the functionality (e.g., GPNMB binding, cell surface expression/localization when included in a CAR, intracellular signaling when included in a CAR, and/or the like) of the parental/reference sequence.

In certain embodiments, CAR variants having one or more amino acid substitutions are provided. Sites of interest for substitutional mutagenesis include the CDRs, framework regions (FRs), one or more intracellular domains (e.g., one or more intracellular signaling domains), and/or the like. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into a CAR of interest and the products screened for a desired activity or features, e.g., retained/improved antigen binding, decreased immunogenicity, improved cell surface expression/localization, improved intracellular signaling, and/or the like.

TABLE 2
Amino Acid Substitutions

| Original | | |
|---|---|---|
| Residue | Exemplary Substitutions | Preferred Substitutions |
| Ala (A) | Val; Leu; Ile | Val |
| Arg I | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

According to some embodiments, an anti-GPNMB antibody of the present disclosure is a humanized antibody. As used herein, a humanized antibody is a recombinant polypeptide that is derived from a non-human (e.g., rabbit, rodent, or the like) antibody and has been modified to contain at least a portion of the framework and/or constant regions of a human antibody. Humanized antibodies also encompass chimeric antibodies and CDR-grafted antibodies in which various regions may be derived from different species. Chimeric antibodies may be antibodies that include a variable region from any source linked to a human constant region (e.g., a human Fc domain). Thus, in chimeric antibodies, the variable region can be non-human, and the constant region is human. CDR-grafted antibodies are antibodies that include the CDRs from a non-human “donor” antibody linked to the framework region from a human “recipient” antibody.

A CAR of the present disclosure may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a serine-glycine linker, such as a serine-glycine linker that includes the amino acid sequence GGGGSGGGGSGGGGS (G₄S)₃ (SEQ ID NO:54). In some embodiments, a linker separates one or more heavy or light chain variable domains, hinge domains, transmembrane domains, co-stimulatory domains, and/or primary signaling domains. In particular embodiments, the CAR includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In certain embodiments, the antigen binding domain of the CAR is followed by one or more spacer domains that moves the antigen binding domain away from the effector cell surface (e.g., the surface of a T cell expressing the CAR) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In one embodiment, the spacer domain includes the CH2 and/or CH3 of IgG1, IgG4, or IgD. Illustrative spacer domains suitable for use in the CARs described herein include the hinge region derived from the extracellular regions of type 1 membrane proteins such as CD8α and CD4, which may be wild-type hinge regions from these molecules or variants thereof. In certain aspects, the hinge domain includes a CD8α hinge region. In some embodiments, the hinge is a PD-1 hinge or CD152 hinge.

The “transmembrane domain” (Tm domain) is the portion of the CAR that fuses the extracellular binding portion and intracellular signaling domain and anchors the CAR to the plasma membrane of the cell (e.g., immune effector cell). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, or PD-1.

In one embodiment, a CAR includes a Tm domain derived from CD8α. In certain aspects, a CAR includes a Tm domain derived from CD8α and a short oligo- or polypeptide linker, e.g., between 1, 2, 3, 4, 5, 6, 7, 8, 9, or 10 amino acids in length, that links the Tm domain and the intracellular signaling domain of the CAR. A glycine-serine linker may be employed as such a linker, for example.

The “intracellular signaling” domain of a CAR refers to the part of a CAR that participates in transducing the signal from CAR binding to a target molecule/antigen into the interior of the immune effector cell to elicit effector cell function, e.g., activation, cytokine production, proliferation and/or cytotoxic activity, including the release of cytotoxic factors to the CAR-bound target cell, or other cellular responses elicited with target molecule/antigen binding to the extracellular CAR domain. Accordingly, the term “intracellular signaling domain” refers to the portion of a protein which transduces the effector function signal and that directs the cell to perform a specialized function. To the extent that a truncated portion of an intracellular signaling domain is used, such truncated portion may be used in place of a full-length intracellular signaling domain as long as it transduces the effector function signal. The term intracellular signaling domain is meant to include any truncated portion of an intracellular signaling domain sufficient for transducing effector function signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a CAR of the present disclosure may include an intracellular signaling domain that includes one or more “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a CAR of the present disclosure include those derived from FcRγ, FcRβ, CD3γ, CD3δ, CD3ε, CD3ζ, CD22, CD79α, CD79β, and CD66δ. In certain embodiments, a CAR includes a CD3 primary signaling domain and one or more costimulatory signaling domains. The intracellular primary signaling and costimulatory signaling domains are operably linked to the carboxyl terminus of the transmembrane domain.

In some embodiments, the CAR includes one or more costimulatory signaling domains to enhance the efficacy and expansion of immune effector cells (e.g., T cells) expressing the CAR. As used herein, the term “costimulatory signaling domain” or “costimulatory domain” refers to an intracellular signaling domain of a costimulatory molecule or an active fragment thereof. Example costimulatory molecules suitable for use in CARs contemplated in particular embodiments include TLR1 through TLR10 (and downstream signaling molecules), CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the CAR includes one or more costimulatory signaling domains selected from the group consisting of 4-1BB (CD137), CD28, and CD134, and a CD3 primary signaling domain.

A CAR of the present disclosure may include any variety of suitable domains including but not limited to a leader sequence; hinge, spacer and/or linker domain(s); transmembrane domain(s); costimulatory domain(s); signaling domain(s) (e.g., CD3ζ domain(s)); ribosomal skip element(s); restriction enzyme sequence(s); reporter protein domains (e.g., a domain comprising a tag, e.g., a MYC tag); and/or the like.

According to some embodiments, a CAR of the present disclosure includes an extracellular domain (e.g., a single chain antibody, such as any of the scFvs described herein) that binds to GPNMB; a transmembrane domain from a polypeptide selected from the group consisting of: CD4, CD8α, CD154, and PD-1; one or more intracellular costimulatory signaling domains from a polypeptide selected from the group consisting of: 4-1BB (CD137), CD28, and CD134; and an intracellular signaling domain from a polypeptide selected from the group consisting of: FcRγ, FcRβ, CD3γ, CD3δ, CD3ε, CD3ζ, CD22, CD79α, CD79β, and CD66δ. Such a CAR may further include a spacer domain between the antigen-binding portion and the transmembrane domain, e.g., a CD8α hinge.

In certain embodiments, a CAR of the present disclosure comprises a CD8α hinge, a CD8α transmembrane domain, a 4-1BB costimulatory domain, and a CD3ζ primary signaling domain. According to some embodiments, provided are CARs that comprise—from N-terminus to C-terminus—a variable light chain (V_{L}) polypeptide of an antibody described herein, a linker, the variable heavy chain (V_{H}) of the antibody, a CD8α hinge region (which in some embodiments is an extended CD8 hinge region), a CD8α transmembrane domain, a 4-1BB costimulatory domain, and a CD3ζ signaling domain. According to certain embodiments, provided are CARs that comprise—from N-terminus to C-terminus—a variable heavy chain (V_{H}) polypeptide of an antibody described herein, a linker, the variable light chain (V_{L}) of the antibody, a CD8α hinge region (which in some embodiments is an extended CD8 hinge region), a CD8α transmembrane domain, a 4-1 BB costimulatory domain, and a CD3ζ signaling domain. Any of the CARs of the present disclosure may include a domain N-terminal to the V_{H} polypeptide. For example, a leader sequence (e.g., a CD8α or GM-CSFR leader sequence) may be present at the N-terminus of a CAR of the present disclosure.

Using the information provided herein, the anti-GPNMB CARs of the present disclosure may be expressed using techniques well known to those of skill in the art. For example, a nucleic acid sequence(s) encoding the amino acid sequence of a CAR of the present disclosure can be used to express the CAR. The amino acid sequences provided herein (see, e.g., Table 1 and the Experimental section (e.g., Table 6) below) can be used to determine appropriate nucleic acid sequences encoding the CARs and the nucleic acids sequences then used to express the CARs. The nucleic acid sequence(s) can be optimized to reflect particular codon “preferences” for various expression systems according to standard methods well known to those of skill in the art. Using the sequence information provided, the nucleic acids may be synthesized according to a number of standard methods known to those of skill in the art.

Once a nucleic acid(s) encoding a subject CAR is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

### Nucleic Acids, Expression Vectors and Cells

In view of the section above regarding methods of expressing the CARs of the present disclosure, it will be appreciated that the present disclosure also provides nucleic acids, expression vectors and cells.

In certain embodiments, provided is a nucleic acid encoding any of the CARs of the present disclosure, e.g., any of such CARs described herein above. Because of the knowledge of the codons corresponding to the various amino acids, availability of an amino acid sequence of a polypeptide of interest provides a description of all the polynucleotides capable of encoding the polypeptide of interest. The degeneracy of the genetic code, where the same amino acids are encoded by alternative or synonymous codons allows an extremely large number of nucleic acids to be made, all of which encode the enzymes disclosed herein. Thus, having identified a particular amino acid sequence, those of ordinary skill in the art could make any number of different nucleic acids by simply modifying the sequence of one or more codons in a way which does not change the amino acid sequence of the polypeptide of interest. In this regard, the present disclosure specifically contemplates each and every possible variation of polynucleotides that could be made by selecting combinations based upon the possible codon choices, and all such variations are to be considered specifically disclosed for any polypeptide disclosed herein, including any of the amino acid sequences of the G1, G2 or G4 scFvs and CARs comprising the same set forth in Tables 1 and 6 herein.

The nucleotide sequences of the nucleic acids of the present disclosure may be codon-optimized. “Codon-optimized” refers to changes in the codons of the polynucleotide encoding a polypeptide to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, a nucleic acid of the present disclosure encoding a polypeptide may be codon-optimized for optimal production from the host organism selected for expression, e.g., human cells, such as human immune cells (e.g., human T cells).

Also provided are expression vectors comprising any of the nucleic acids of the present disclosure. Expression of natural or synthetic nucleic acids encoding the CARs of the present disclosure can be achieved by operably linking a nucleic acid encoding the CAR to a promoter (which is either constitutive or inducible) and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

Also provided are cells that include any of the nucleic acids and/or expression vectors of the present disclosure. In certain aspects, provided are cells where the CAR is expressed on the surface of the cell. By “expressed on the surface of the cell” is meant the CAR has been trafficked to the cell membrane such that—in the case of a CAR—the extracellular binding domain is displayed on the cell surface, the transmembrane portion passes through the cell membrane, and the one or more intracellular signaling domains are disposed on the intracellular side of the cell membrane. Upon binding of the extracellular binding domain to GPNMB, the intracellular signaling domain of the CAR participates in transducing the signal from the binding into the interior of the cell (e.g., an effector cell, such as a T cell, to elicit effector cell function).

In some embodiments, the cells are eukaryotic cells. Eukaryotic cells of interest include, but are not limited to, yeast cells, insect cells, mammalian cells, and the like. Mammalian cells of interest include, e.g., murine cells, non-human primate cells, human cells, and the like.

“Recombinant host cells,” “host cells,” “cells,” “cell lines,” “cell cultures,” and other such terms denoting microorganisms or higher eukaryotic cell lines, refer to cells which can be, or have been, used as recipients for a recombinant vector or other transferred DNA, and include the progeny of the cell which has been transfected. Host cells may be cultured as unicellular or multicellular entities (e.g., tissue, organs, or organoids) including an expression vector of the present disclosure.

In one aspect, the cells provided herein include immune cells. Non-limiting examples of immune cells which may comprise any of the nucleic acids and/or expression vectors of the present disclosure include T cells, B cells, natural killer (NK) cells, macrophages, monocytes, neutrophils, dendritic cells, mast cells, basophils, eosinophils, and hematopoietic stem cells. In some embodiments, the immune cell comprises a T cell. Examples of T cells include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17) CD4+ T cells, CD8+ T cells, virus-specific T cells, alpha beta T cells (T_{αβ}), and gamma delta T cells (T_{γδ}).

In some instances, a cell expressing a CAR of the present disclosure is a stem cell. Non-limiting examples of stem cells of the present disclosure include hematopoietic stem cells (HSCs), induced pluripotent stem cells (iPSCs) or derivatives thereof, and the like.

Also provided are methods of making the cells of the present disclosure. In some embodiments, such methods include transfecting or transducing cells with a nucleic acid or expression vector of the present disclosure. The term “transfection” or “transduction” is used to refer to the introduction of foreign DNA into a cell. A cell has been “transfected” when exogenous DNA has been introduced inside the cell membrane. A number of transfection techniques are generally known in the art. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material.

In some embodiments, a cell of the present disclosure is produced by transfecting the cell with a viral vector encoding the CAR. In some embodiments, the cell is a T cell, such that provided are methods of producing CAR T-cells. In some embodiments, such methods include activating a population of T cells (e.g., T cells obtained from an individual to whom a CAR T-cell therapy will be administered), stimulating the population of T cells to proliferate, and transducing the T cell with a viral vector encoding the CAR. In some embodiments, the T cells are transduced with a retroviral vector, e.g., a gamma retroviral vector or a lentiviral vector, encoding the CAR. In some embodiments, the T cells are transduced with a lentiviral vector encoding the CAR.

According to some embodiments, a cell of the present disclosure is produced by directed integration via a genome editing technology such as one employing a CRISPR/Cas9 nuclease, a TALEN nuclease, a ZFN, or the like. For example, a CRISPR-Cas protein, such as e.g., a Cas9 protein, or a polynucleotide encoding a CRISPR-Cas protein and guide RNA (gRNA) or a polynucleotide encoding gRNA, may be employed. As used herein, the term “gRNA” generally encompasses either two-component guide systems (e.g., two gRNAs) as well as single guide RNA (sgRNA) systems, unless inappropriate and/or denoted otherwise. In some instances, the gRNA or multiple gRNAs may be configured and employed to target a desired locus as described herein or one or more elements thereof such as one of more exons of a gene present at the locus. For example, in some instances, a gRNA or multiple gRNAs may be configured and employed to target a locus or one or more elements thereof, such as e.g., one or more exons of the locus.

In certain embodiments, directed integration may include the use of a Cas9 nuclease, including natural and engineered Cas9 nucleases, as well as nucleic acid sequences encoding the same. Useful Cas9 nucleases include but are not limited to e.g., Streptococcus pyogenes Cas9 and variants thereof, Staphylococcus aureus Cas9 and variants thereof, Actinomyces naeslundii Cas9 and variants thereof, Cas9 nucleases also include those discussed in PCT Publications Nos. WO 2013/176772 and WO2015/103153 and those reviewed in e.g., Makarova et al. (2011) Nature Reviews Microbiology 9:467-477, Makarova et al. (2011) Biology Direct 6:38, Haft et al. (2005) PLOS Computational Biology 1:e60 and Chylinski et al. (2013) RNA Biology 10:726-737, the disclosures of which are incorporated herein by reference in their entirety. In some instances, a non-Cas9 CRISPR nuclease (or engineered variant thereof) may be employed, including but not limited to e.g., Cpf1 or Cpf1 variant.

The CRISPR system offers significant versatility in gene editing in part because of the small size and high frequency of necessary sequence targeting elements within host genomes. CRISPR guided Cas9 nuclease requires the presence of a protospacer adjacent motif (PAM), the sequence of which depends on the bacteria species from which the Cas9 was derived (e.g. for Streptococcus pyogenes the PAM sequence is “NGG”) but such sequences are common throughout various target nucleic acids. The PAM sequence directly downstream of the target sequence is not part of the guide RNA but is obligatory for cutting the DNA strand. Synthetic Cas9 nucleases have been generated with novel PAM recognition, further increasing the versatility of targeting, and may be used in the methods described herein. Cas9 nickases (e.g., Cas9 (D10A) and the like) that cleave only one strand of target nucleic acid as well as endonuclease deficient (i.e., “dead”) dCas9 variants with additional enzymatic activities added by an attached fusion protein have also been developed.

In certain embodiments, directed integration may be performed by base-editing strategies, e.g., as described in U.S. Patent Application Publication No. US 2022/0220462; or prime editing strategies, e.g., as described in U.S. Pat. No. 11,447,770; the disclosures of which are incorporated herein by reference in their entireties for all purposes.

Cells of the present disclosure may be autologous/autogeneic (“self”) or non-autologous (“non-self,” e.g., allogeneic, syngeneic, or xenogeneic). “Autologous” as used herein, refers to cells from the same individual. “Allogeneic” as used herein refers to cells of the same species that differ genetically from the cell in comparison. “Syngeneic,” as used herein, refers to cells of a different individual that are genetically identical to the cell in comparison. In some embodiments, the cells are T cells obtained from a mammal. In some embodiments, the mammal is a primate. In some embodiments, the primate is a human.

T cells may be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In certain embodiments, T cells can be obtained from a unit of blood collected from an individual using any number of known techniques such as sedimentation (e.g., FICOLL™ separation), aphaeresis, or any other convenient approach.

In some embodiments, an isolated or purified population of T cells is used. In some embodiments, T_{CTL} and T_{H} lymphocytes are purified from PBMCs. In some embodiments, the T_{CTL} and T_{H} lymphocytes are sorted into naïve (T_{N}), memory (T_{MEM}), and effector (T_{EFF}) T cell subpopulations either before or after activation, expansion, and/or genetic modification. Suitable approaches for such sorting are known and include, e.g., magnetic-activated cell sorting (MACS), where TN are CD45RA⁺ CD62L⁺ CD95⁻; TSCM are CD45RA⁺ CD62L⁺ CD95⁺; TCM are CD45RO⁺ CD62L⁺ CD95⁺; and TEM are CD45RO⁺ CD62L⁻ CD95⁺. An example approach for such sorting is described in Wang et al. (2016) Blood 127(24):2980-90. According to some embodiments, the isolated or purified population of T cells is a population of T_{REGs} purified from PBMCs.

A specific subpopulation of T cells expressing one or more of the following markers: CD3, CD4, CD8, CD28, CD45RA, CD45RO, CD62, CD127, and HLA-DR can be further isolated by positive or negative selection techniques. In some embodiments, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In some embodiments, the manufactured T cell compositions do not express one or more of the following markers: CD57, CD244, CD 160, PD-1, CTLA4, TIM3, and LAG3. In some embodiments, the manufactured T cell compositions do not substantially express one or more of the following markers: CD57, CD244, CD 160, PD-1, CTLA4, TIM3, and LAG3.

In order to achieve therapeutically effective doses of T cell compositions, the T cells may be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes. In some embodiments, T cells are activated and expanded for about 1 to 21 days, e.g., about 5 to 21 days. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of a nucleic acid (e.g., expression vector) encoding the polypeptide into the T cells.

In some embodiments, T cells are activated and expanded for about 6 hours, about 12 hours, about 18 hours or about 24 hours prior to introduction of a nucleic acid (e.g., expression vector) encoding the CAR the into the T cells. In some embodiments, T cells are activated at the same time that a nucleic acid (e.g., an expression vector) encoding the CAR is introduced into the T cells.

In some embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

In some embodiments, the nucleic acid (e.g., an expression vector) encoding the CAR is introduced into the cell (e.g., a T cell) by microinjection, transfection, a genome editing technology (e.g., employing a CRISPR/Cas9 nuclease, a TALEN nuclease, a ZFN, or the like), lipofection, heat-shock, electroporation, transduction, gene gun, microinjection, DEAE-dextran-mediated transfer, and the like. In some embodiments, the nucleic acid (e.g., expression vector) encoding the CAR is introduced into the cell (e.g., a T cell) by AAV transduction. The AAV vector may comprise ITRs from AAV2, and a serotype from any one of AAV1, AAV2, AAV3, AAV4, AAV5, AAV6, AAV7, AAV8, AAV9, or AAV 10. In some embodiments, the AAV vector comprises ITRs from AAV2 and a serotype from AAV6. In some embodiments, the nucleic acid (e.g., expression vector) encoding the CAR is introduced into the cell (e.g., a T cell) by lentiviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV). The lentiviral vector may be integration competent or an integrase deficient lentiviral vector (TDLV). In one embodiment, IDLV vectors including an HIV-based vector backbone (i.e., HIV cis-acting sequence elements) are employed.

Also provided are viruses that include any of the nucleic acids and/or expression vectors of the present disclosure.

### Compositions

In other aspects, provided herein are compositions comprising any of the nucleic acids, expression vectors, and/or cells of the present disclosure.

In some embodiments, the compositions include any of the nucleic acids, expression vectors, and/or cells of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

Compositions comprising any of the cells of the present disclosure suitable for administration to human subjects are also provided. Such compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of condition associated with GPNMB expression and/or activity, as compared to a control. An effective amount can be administered in one or more administrations.

The cells of the present disclosure can be incorporated into a variety of formulations for therapeutic administration. In some embodiments, the cells of the present disclosure are formulated into pharmaceutical compositions by combination with appropriate, pharmaceutically acceptable excipients or diluents.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

### Kits

Aspects of the present disclosure further include kits. In certain embodiments, the kits find use in producing the cells of the present disclosure, non-limiting examples of which include CAR T-cells expressing and anti-GPNMB CAR of the present disclosure on the surface thereof.

Accordingly, in certain embodiments, a kit of the present disclosure comprises any of nucleic acids and/or expression vectors of the present disclosure, and instructions for transducing cells (e.g., T cells) with the nucleic acid and/or expression vector. As will be appreciated, the kits of the present disclosure may include any of the features described above in the sections relating to the subject nucleic acids and expression vectors, which are not reiterated herein for purposes of brevity.

Components of the kits may be present in separate containers, or multiple components may be present in a single container. A suitable container includes a single tube (e.g., vial), one or more wells of a plate (e.g., a 96-well plate, a 384-well plate, etc.), or the like.

The instructions included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods of Use

Aspects of the present disclosure further include methods of using cells expressing the CARs of present disclosure. The methods are useful in a variety of contexts, including in vitro and/or in vivo research and/or clinical applications.

In certain embodiments, provided are methods of treating a condition associated with GPNMB expression and/or activity in a subject in need thereof, the methods comprising administering an effective amount of a composition comprising cells expressing the CAR (e.g., CAR T-cells, CAR NK cells, or the like) to the subject.

According to some embodiments, the condition associated with GPNMB expression and/or activity is cancer. The subject methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. The cancer may be characterized by cancer cells that express GPNMB on the surface thereof (GPNMB+ cancer cells). In certain embodiments, the cancer comprises a solid tumor. According to some embodiments, the solid tumor is a sarcoma, carcinoma, lymphoma, or blastoma. In some embodiments, when the cancer comprises a solid tumor, the cancer is characterized by non-cancer cells in the tumor microenvironment (TME) that express GPNMB on the surface thereof. Non-limiting examples of non-cancer cells exhibiting cell surface expression of GPNMB in the TME include immune cells (e.g., macrophages), endothelial cells, fibroblasts (e.g., cancer associated fibroblasts (CAFs)), etc.

Examples of cancers that may be treated using the methods of the present disclosure include, but are not limited to, sarcoma, carcinoma, lymphoma, and blastoma. More particular examples of such cancers include renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgendependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

In certain embodiments, the cancer is a sarcoma. Non-limiting examples of sarcomas treatable using the methods of the present disclosure include soft-tissue sarcomas (STSs). In some embodiments, when the sarcoma is a STS, the STS is alveolar soft tissue sarcoma (ASPS). In certain embodiments, the subject comprises metastatic cancer. For example, the subject may comprise central nervous system (CNS) metastasis.

According to some embodiments, the cancer is a carcinoma. In some instances, when the cancer is a carcinoma, the carcinoma is basal cell carcinoma, squamous cell carcinoma, renal cell carcinoma, ductal carcinoma in situ (DCIS), invasive ductal carcinoma, or adenocarcinoma.

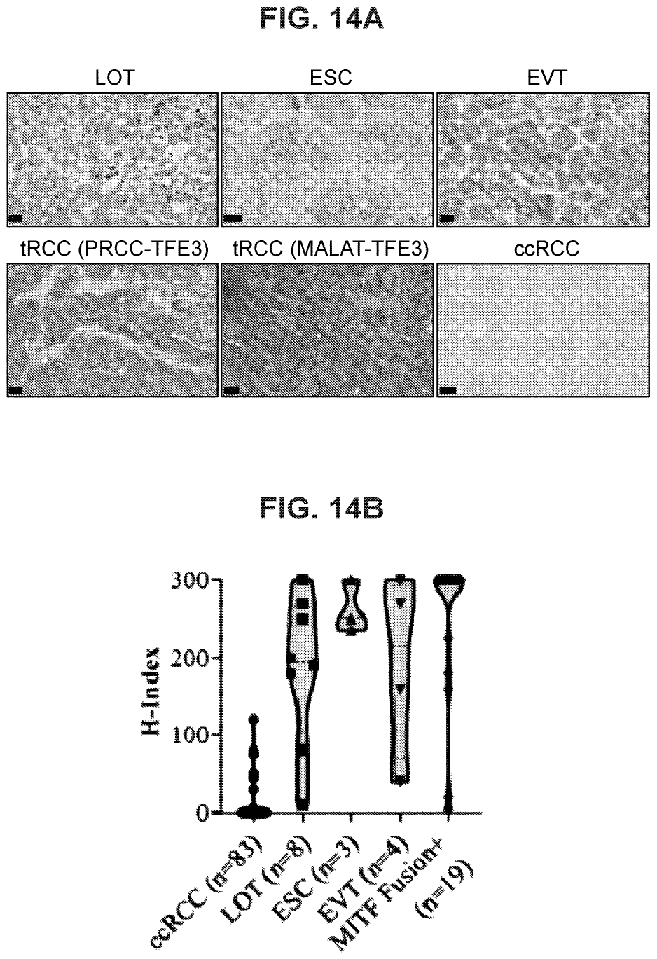

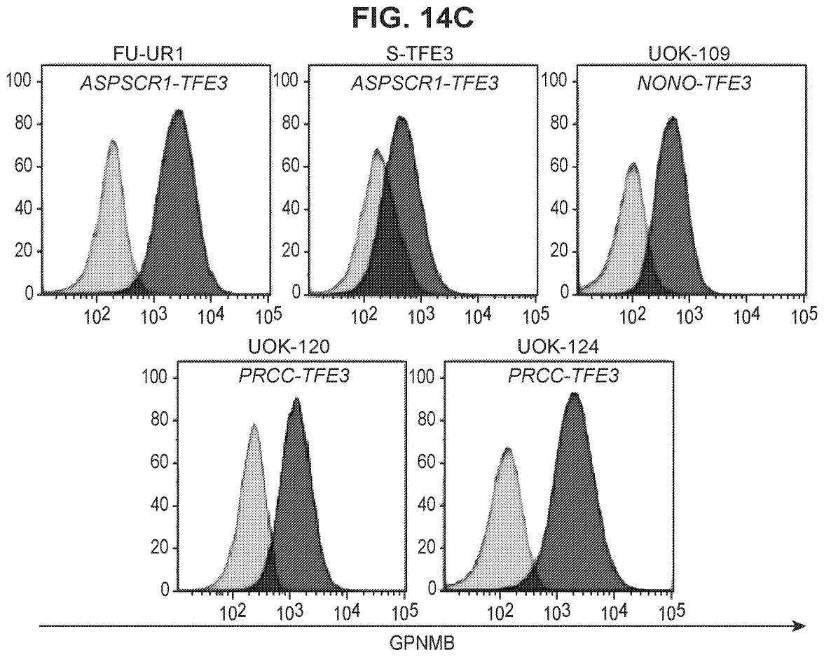

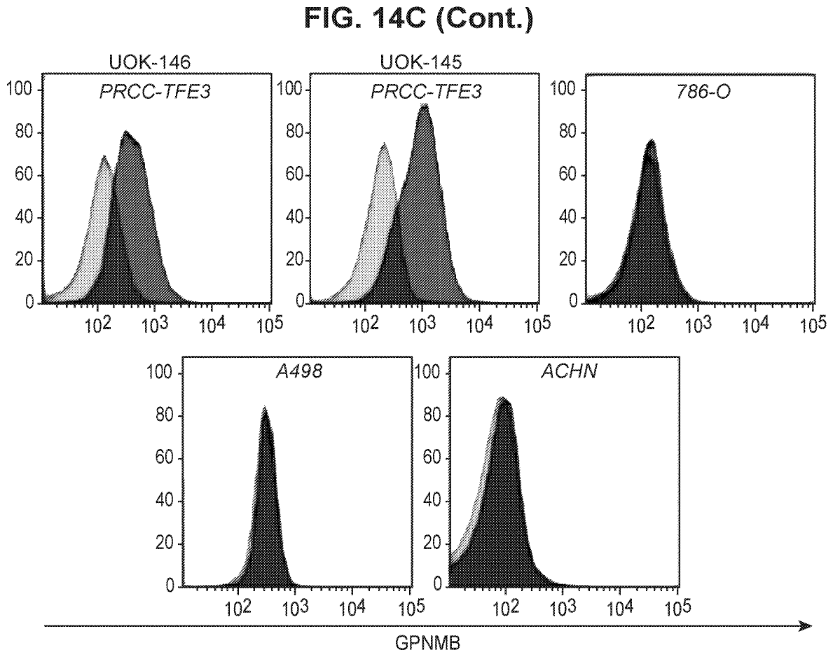

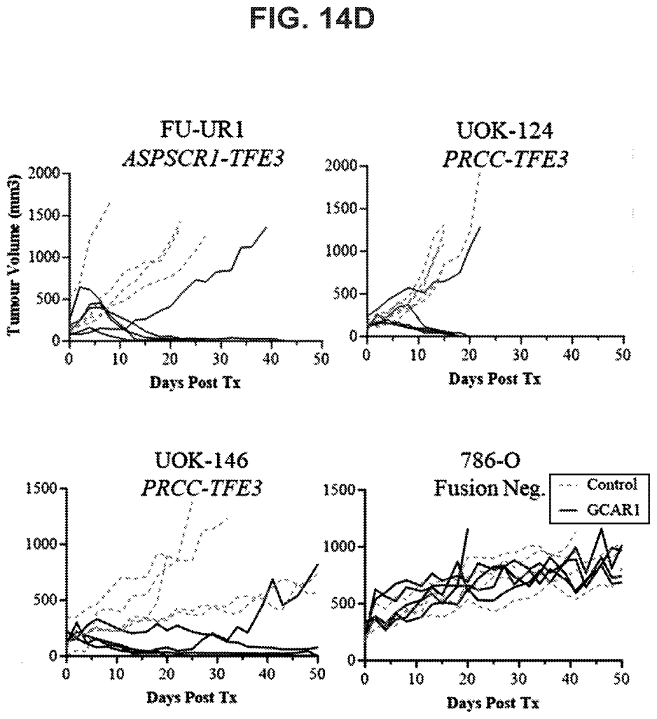

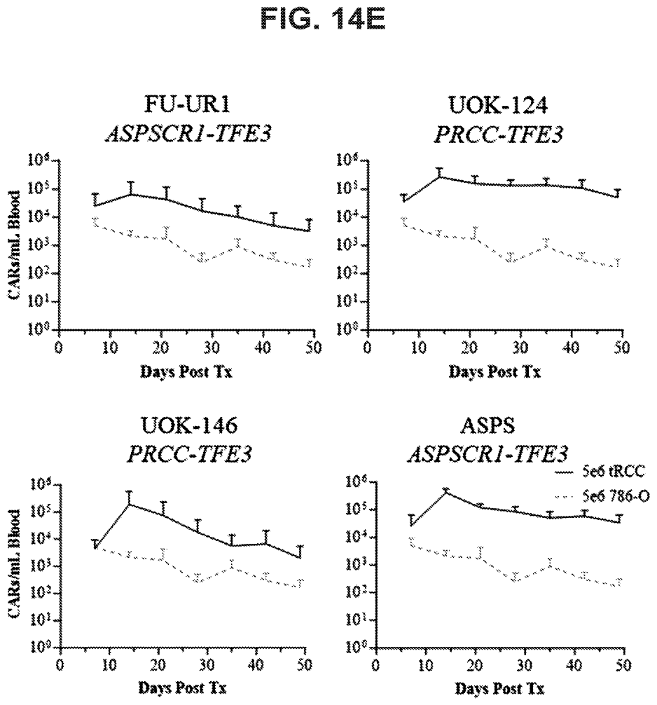

In certain embodiments, the cancer is renal cell carcinoma (e.g., translocation renal cell carcinoma (tRCC) or TSC/mTOR-altered RCC (see Salles et al. (2022) J Pathol. 257(2):158-171; FIG. 14a,b), breast cancer (e.g., triple-negative breast cancer (TNBC)), melanoma, or a soft-tissue sarcoma (e.g., ASPS). In some instances, the cancer is tRCC. According to some embodiments, the cancer is GPNMB+ TNBC. In certain embodiments, the cancer is ASPS.

In some instances, the subject has a cancer comprising a fusion protein involving a microphthalmia family transcription factor. Such transcription factors include MITF, TFEB, TFE3, and TFEC. GPNMB is known to be expressed on the surface of cancer cells comprising such fusions. See, e.g., Salles et al. (2022) J Pathol. 257(2):158-171. See also FIG. 14a,b.

According to some embodiments, the subject has a cancer characterized by activation of one or more microphthalmia family transcription factors, e.g., MITF, TFEB, TFE3, and/or TFEC. GPNMB is known to be expressed on the surface of cancer cells comprising activation of such transcription factors. See, e.g., Hong et al. (2010) PLoS One 5(12):e15793; and Salles et al. (2022) J Pathol. 257(2):158-171). See also FIG. 14a,b.

In certain instances, the condition associated with GPNMB expression and/or activity is a neurodegenerative disease. Non-limiting examples of neurodegenerative diseases treatable by the methods of the present disclosure include Alzheimer's disease (AD), Gaucher disease, Niemann-Pick Type C disease, amyotrophic lateral sclerosis (ALS), Parkinson's disease (PD), amyloidosis, and the like.

The cells (e.g., CAR T-cells) may be administered via any suitable route of administration, e.g., parenteral (e.g., by intravenous, intra-arterial, subcutaneous, intramuscular, or epidural injection, intra-tumoral administration, intra-cerebral administration), or the like.

The cells (e.g., CAR T-cells) may be administered in a composition in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a cancer or neurodegenerative disease, as compared to a control. With respect to cancer, in some embodiments, the therapeutically effective amount is sufficient to slow the growth of a tumor, reduce the size of a tumor, and/or the like. An effective amount can be administered in one or more administrations.

As described above, aspects of the present disclosure include methods for treating a condition associated with GPNMB expression and/or activity. By treatment is meant at least an amelioration of one or more symptoms associated with the condition of the individual, where amelioration is used in a broad sense to refer to at least a reduction in the magnitude of a parameter (e.g. symptom) associated with the condition being treated. As such, treatment also includes situations where the condition, or at least one or more symptoms associated therewith, are completely inhibited, e.g., prevented from happening, or stopped, e.g., terminated, such that the individual no longer suffers from the condition, or at least the symptoms that characterize the condition.

The cells (e.g., CAR T-cells) may be administered to the individual alone or in combination with a second agent. Second agents of interest include, but are not limited to, agents approved by the United States Food and Drug Administration and/or the European Medicines Agency (EMA) for use in treating cancer, e.g., sarcomas, such as soft tissue sarcomas, e.g., ASPS. In some embodiments, the second agent is an immune checkpoint inhibitor. Immune checkpoint inhibitors of interest include, but are not limited to, a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When the cells (e.g., CAR T-cells) are administered with a second agent, the cells may be administered to the individual according to any suitable administration regimen. According to certain embodiments, the cells and the second agent are administered according to a dosing regimen approved for individual use. In some embodiments, the administration of the cells permits the second agent to be administered according to a dosing regimen that involves one or more lower and/or less frequent doses, and/or a reduced number of cycles as compared with that utilized when the second agent is administered without administration of the cells. In certain embodiments, the administration of the second agent permits the cells to be administered according to a dosing regimen that involves one or more lower and/or less frequent doses, and/or a reduced number of cycles as compared with that utilized when the cells are administered without administration of the second agent.

In some embodiments, one or more doses of the cells and the second agent are administered concurrently to the subject. By “concurrently” is meant the cells and the second agent are either present in the same composition, or the cells and the second agent are administered as separate pharmaceutical compositions within 1 hour or less, 30 minutes or less, or 15 minutes or less.

In some embodiments, one or more doses of the cells and the second agent are administered sequentially to the subject.

In some embodiments, the cells are administered to the subject in different compositions and/or at different times. For example, the cells may be administered prior to administration of the second agent, e.g., in a particular cycle. Alternatively, the second agent may be administered prior to administration of the cells, e.g., in a particular cycle. The second agent to be administered may be administered a period of time that starts at least 1 hour, 3 hours, 6 hours, 12 hours, 24 hours, 48 hours, 72 hours, or up to 5 days or more after the administration of the first agent to be administered.

In some embodiments, administration of one agent is specifically timed relative to administration of the other agent. For example, in some embodiments, the cells are administered so that a particular effect is observed (or expected to be observed, for example based on population studies showing a correlation between a given dosing regimen and the particular effect of interest).

According to some embodiments, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular individual of interest. The cells and the second agent may be administered together or independently via any suitable route of administration. The cells and the second agent may be administered via a route of administration independently selected from oral, parenteral (e.g., by intravenous, intra-arterial, subcutaneous, intramuscular, or epidural injection), topical, intra-nasal, intra-tumoral administration, or the like.

The following examples are offered by way of illustration and not by way of limitation.

### EXPERIMENTAL

### Example 1—ASPS Gene Expression Profiling

Soft-tissue sarcomas (STS) are a rare and diverse group of cancers derived from cells of primitive mesenchymal origin. ~30% of STS, but most pediatric and AYA cases, are molecularly characterized by a recurrent, balanced chromosomal translocation leading to the formation of a novel oncogene. These fusions involve transcription factors and/or chromatin remodelling complexes that are able to drive transformation and tumorigenesis, often in the absence of few other genetic abnormalities. Despite the current understanding of how pathognomonic fusions drive STS etiology, many STS subtypes remain without a standard-of care treatment and are ultimately fatal, particularly in the setting of recurrent and/or metastatic disease.

Alveolar soft tissue sarcoma (ASPS) is cytogenetically characterized by a t(X:17)(p11;q25) translocation that fuses the third or fourth exon of the TFE3 transcription factor with the first seven exons of the DNA binding protein ASPSCR1. ASPS has an indolent disease course, often presenting as a painless mass in the lower extremities. Asymptomatic presentation can delay diagnosis, resulting in approximately half of patients presenting with metastasis, typically to the lung, bone and/or brain. Recurrence in patients who present with metastases is inevitable, and the limited systemic treatments leaves only metastasectomy, if possible, as a life extending intervention. All patients with metastases eventually succumb to their disease.

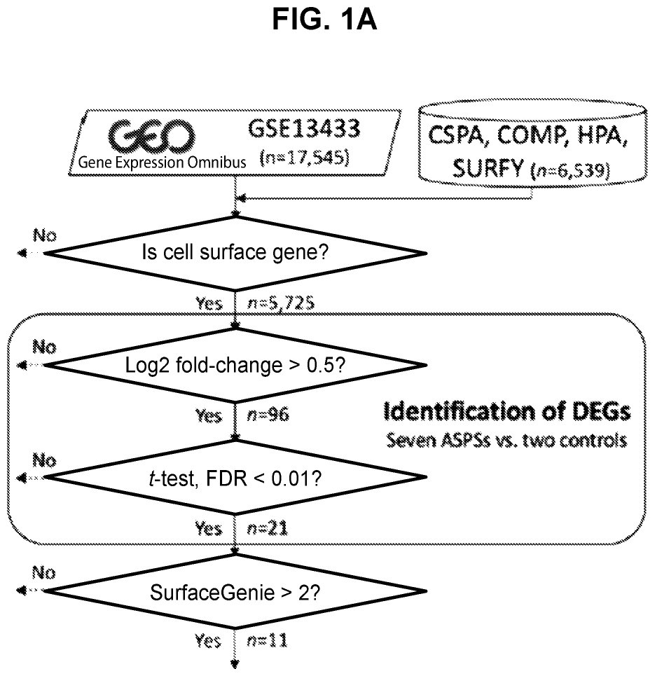

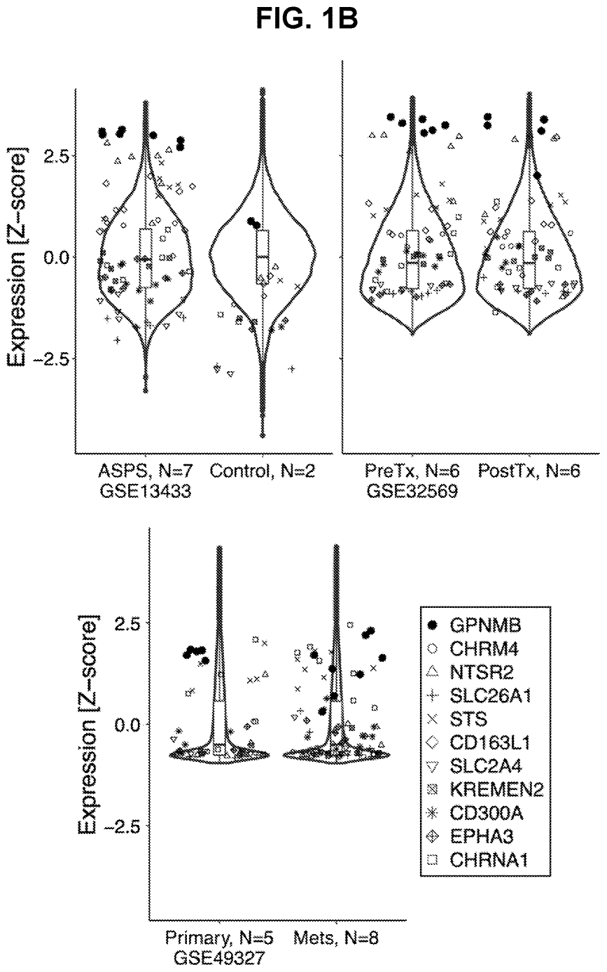

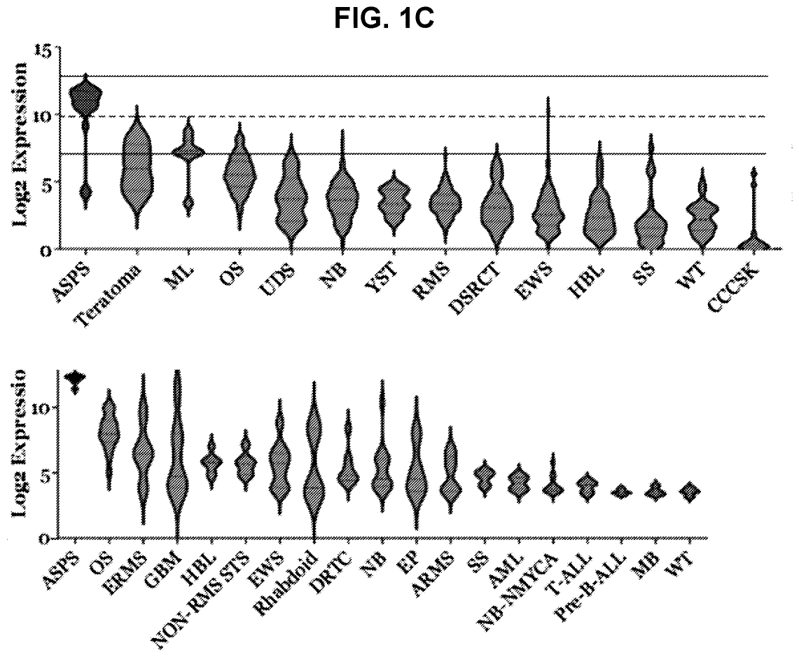

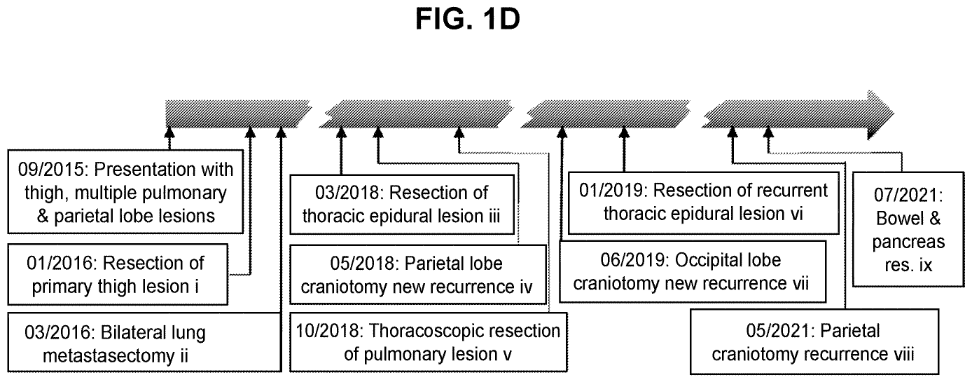

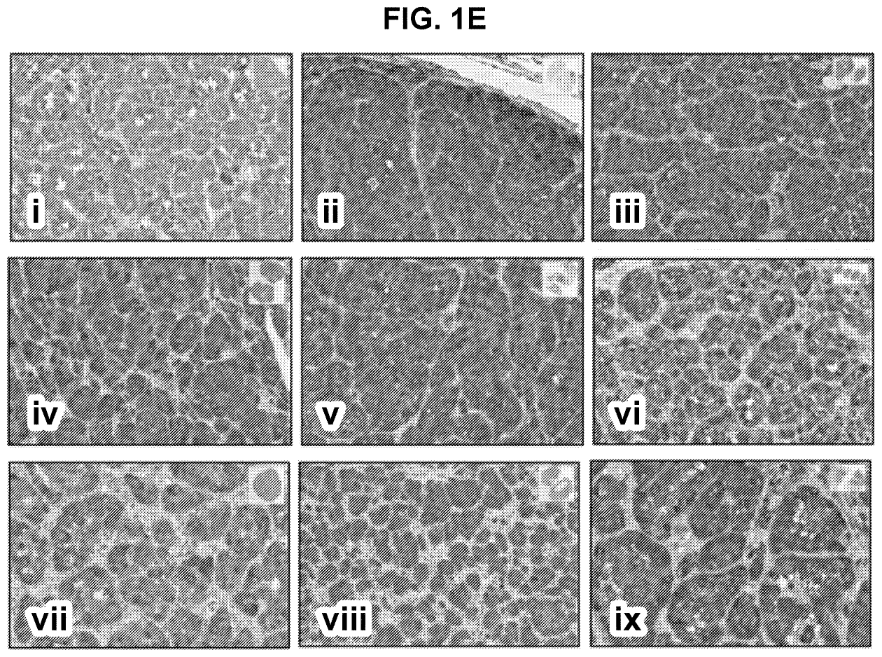

To analyze the potential surfaceome of ASPS, a differential expression analysis with the gene expression profile of ASPS was performed (FIG. 1a). Eleven genes were identified as ‘ASPS-specific genes’ that are likely expressed on the cell surface membrane (Table 3). GPNMB was reported as the most statistically significant genes (lowest FDR; FDR=0.0001, Log 2-FC=0.6044) while SLC2A4 was the highest regulated ASPS-specific gene (greatest fold-change; FDR=0.0003, Log 2-FC=1.1520). Using these 11 ASPS-specific genes from this cohort, the level of expression was investigated in multiple ASPS samples (FIG. 1b). It was observed that GPNMB was highly expressed in every ASPS sample from three cohorts. NTSR2, CHRNA1, and STS appeared to be the data-specific DE genes, with the remaining candidates were below or close to the median expression. Focusing in on GPNMB, the expression was examined across several publicly available gene expression datasets, comparing GPNMB expression across other paediatric and AYA tumor types. GPNMB was found to be expressed to high levels in all ASPS samples analysed (FIG. 1c). As GPNMB expression is regulated by the Microphthalmia family of basic helix-loop-helix leucine zipper transcription factors (MiTF/TFE), of which TFE3 is a member, it is likely that the high and universal GPNMB expression in ASPS is driven by the ASPSCR1-TFE3 fusion. Indeed, this has been demonstrated in Xp11-fusion driven renal cell carcinomas, where Xp11-fusion partners, including ASPSCR1, have been shown to drive GPNMB expression in these cancers (Tanaka et al. (2017) Cancer Res 77:897-907; Baba et al. (2019) Mol Cancer Res 17:1613-1626) as also demonstrated in a subsequent example.

TABLE 3
Eleven candidate genes that are ASPS-specific and cell surface proteins.

| | | | | | | Surfaceome | Surface |
|---|---|---|---|---|---|---|---|
| Gene | Log2-FC | P-value | FDR | UniProfID | EnsemblGeneID | DBS | Genie |
| GPNMB | 0.6044 | 1.55E−06 | 0.00011 | Q14956 | ENSG00000136235 | CSPA-COMP | 3 |
| CHRM4 | 0.7259 | 4.41E−05 | 0.00065 | P08173 | ENSG00000180720 | COMP | 4 |
| NTSR2 | 0.8538 | 6.89E−05 | 0.00086 | O95665 | ENSG00000169006 | COMP | 3 |
| SLC26A1 | 0.6226 | 7.42E−05 | 0.00091 | Q9H2B4 | ENSG00000145217 | COMP | 4 |
| STS | 0.7791 | 8.73E−05 | 0.00102 | P08842 | ENSG00000101846 | CSPA-COMP | 3 |
| CD163L1 | 0.6849 | 0.00016 | 0.00160 | Q9NR16 | ENSG00000177675 | COMP | 4 |
| SLC2A4 | 1.1520 | 0.00030 | 0.00247 | P14672 | ENSG00000181856 | CSPA-COMP | 3 |
| KREMEN2 | 0.5373 | 0.00039 | 0.00298 | Q8NCW0 | ENSG00000131650 | COMP | 4 |
| CD300A | 0.5172 | 0.00065 | 0.00427 | Q9UGN4 | ENSG00000167851 | HPA-COMP | 4 |
| EPHA3 | 0.5342 | 0.00120 | 0.00671 | P29320 | ENSG00000044524 | HPA-CSPA- | 3 |
| | | | | | | COMP | |
| CHRNA1 | 0.6055 | 0.00213 | 0.00997 | P02708 | ENSG00000138435 | HPA-COMP | 3 |

### Example 2—Validation of GPNMB Expression in Clinical ASPS Samples

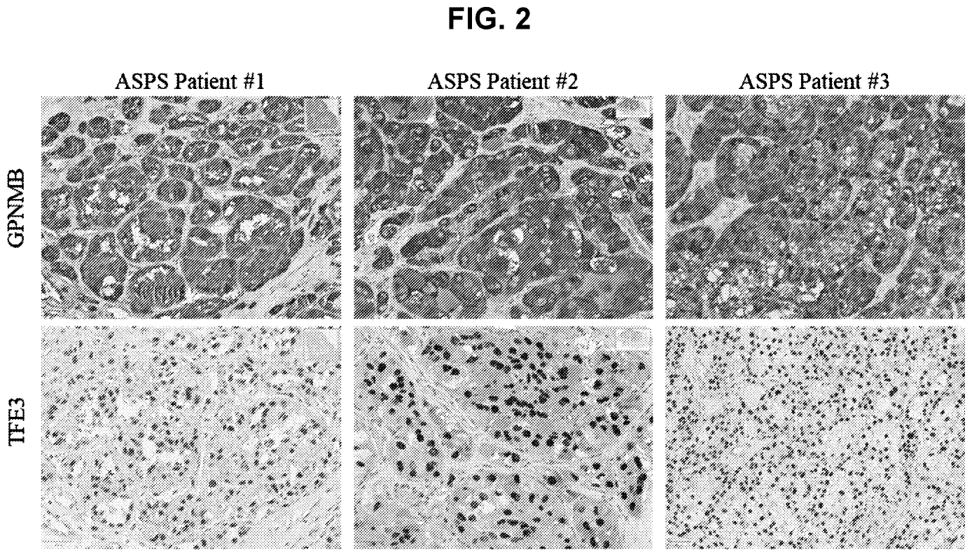

To validate GPNMB expression in clinical ASPS samples, three primary ASPS resections were stained. These samples were clinically diagnosed as ASPS, which was confirmed through assessment of nuclear localization of TFE3. High and homogenous expression of GPNMB was found in these primary tumor sections (FIG. 2). One of the subjects was originally diagnosed with metastatic disease at presentation, and has since had multiple metastasectomies to remove recurrences in the lungs, brain, spine and gastrointestinal tract (FIG. 1d). Staining GPNMB homogenous cell surface expression was observed in all surgical samples, demonstrating the spatial and temporal stability of the potential target (FIG. 1e).

### Example 3—Evaluation of GPNMB as a CAR Target

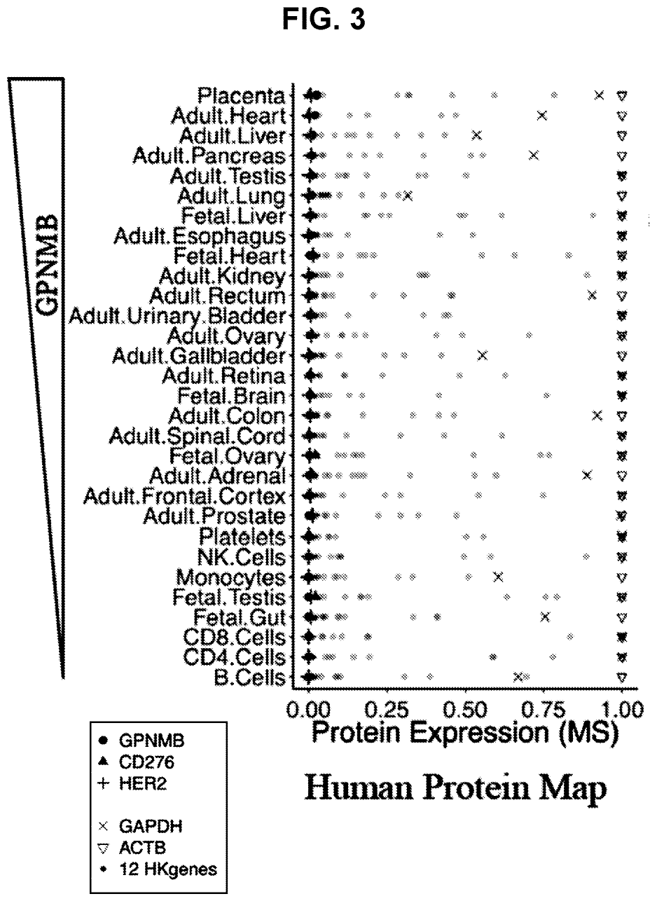

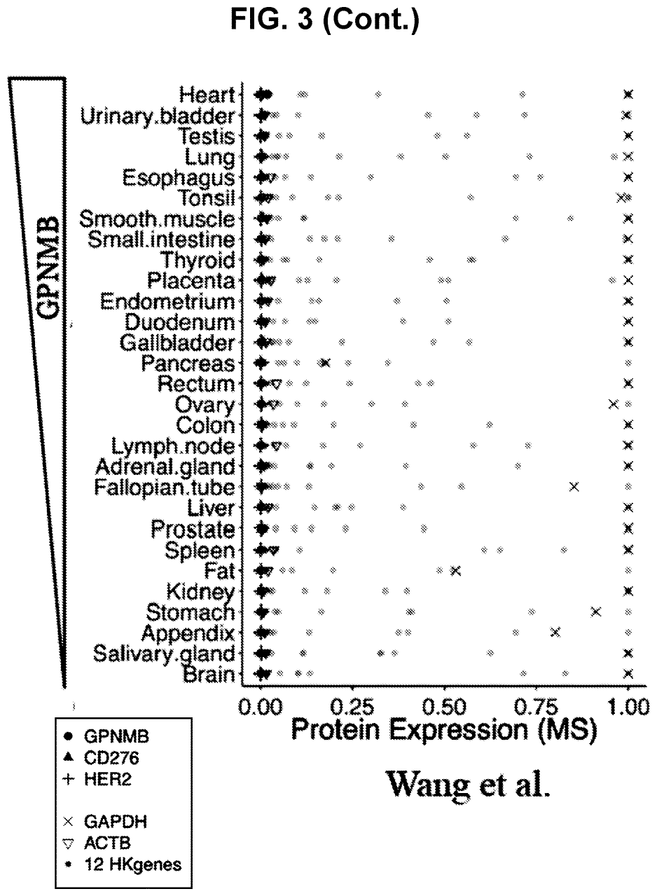

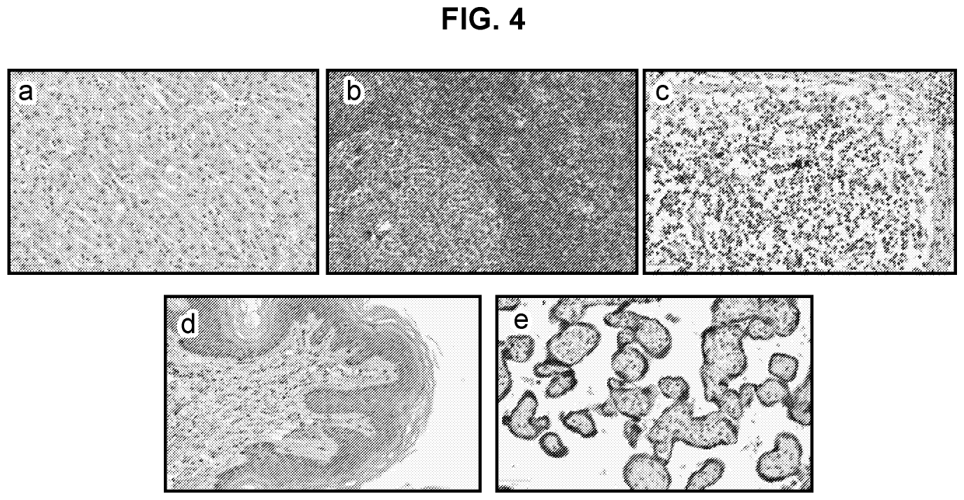

GPNMB has been described to have varied expression across several normal human tissues, but is predominantly expressed in the skin, bone, and several subsets of myeloid-derived cells. Further, GPNMB can occur in diverse cellular locations, such as the cell surface, intracellular vesicles and as a secreted product. To deconvolute human versus mouse protein expression data from the literature, two publicly available mass-spec-based proteomics databases (Kim et al. (2014) Nature 509:575-81; Wang et al. (2019) Mol Syst Biol 15, e8503) were first evaluated, and the expression of GPNMB against other known CAR targets for sarcoma (HER2 and CD276) were evaluated against 14 housekeeping genes (FIG. 3). Housekeeping genes showed a wide range of expression at the protein level and GAPDH and ACTB expression were high across tissues/databases. HER2 and CD276 demonstrated relatively low expression as expected. GPNMB demonstrated comparably low or no expression in all normal tissues, suggesting that GPNMB would be a safe CAR target. However, it was found that both databases had “heart” as amongst the top tissue expressors. The expression of GPNMB was further examined in 34 normal tissues across two normal tissue microarrays. Most tissue had little to no expression, but significant staining (≥2 histology score) was noted in the skin, heart, placenta, and lymphoid tissues (Table 4); however, apart from the placenta and tonsil, GPNMB expression in these tissues was confined within the cytosol (FIG. 4).

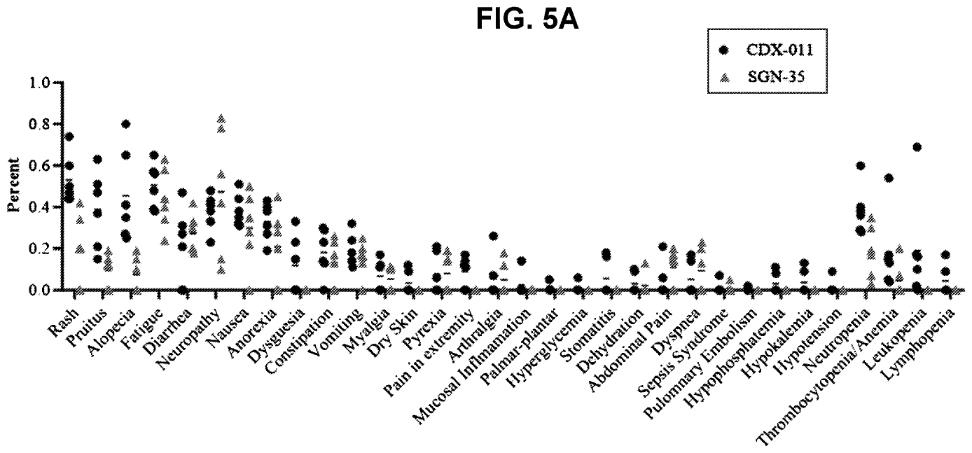

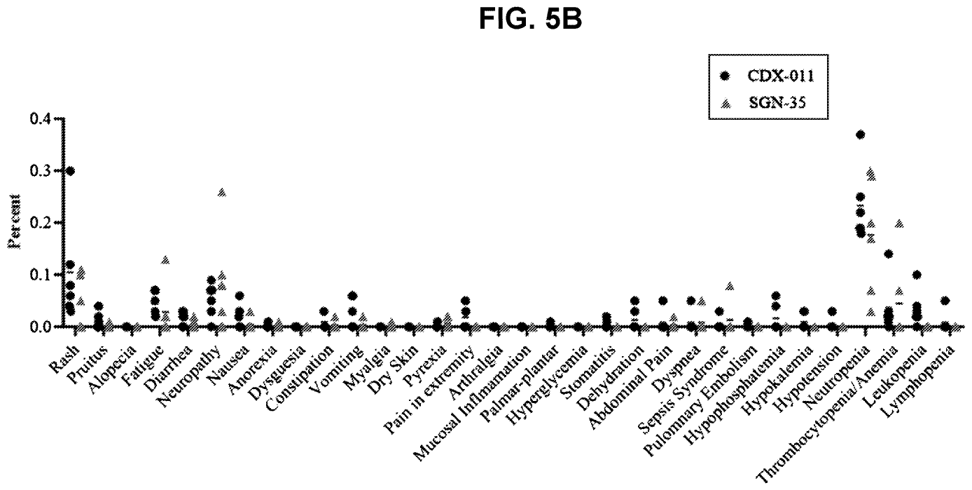

To further evaluate the potential safety of a GPNMB CAR, the adverse events associated with Glembatumumab vedotin (CDX-011) were investigated. CDX-011 was a human GPNMB targeting antibody-drug conjugate clinically evaluated in over six hundred patients with melanoma, breast cancer, lung cancer or osteosarcoma (Table 5). CDX-011 was considered well or generally well tolerated in all these trials, including the NCT02487979, which largely contained paediatric and AYA patients. CDX-011 is bound with a proteolytically cleaved monomethylauristatin E (MMAE), similarly to Brentuximab vedotin (SGN-35), an FDA-approved therapy targeting CD30 in Hodgkin's and T-cell lymphoma. The adverse event profiles of CDX-011 and SGN-35 were directly compared in an effort to separate toxicities attributed to cytotoxic targeting normal GPNMB+ cells versus non-specific toxicity attributed to decoupled MMAE. In the six comparable trials that employed identical dosing strategies and toxicity grading, it was found that amongst all adverse events (AEs) there was a significant increase in skin-related toxicities (rash 53% vs. 19%; pruitus 36% vs. 10%; alopecia 49% vs. 7%) and neutropenia (40% vs. 19%) with CDX-011. Isolating the serious Aes (≥3) alone, only rash (11% vs. 4%) remained significantly different (FIG. 5). Importantly, there was an absence of any cardiac toxicities in the CDX-011 trials.

TABLE 4
Summary Normal Tissue Array ≥2

| Tissue | Array | Staining (0-3) | Cells staining | Stain location |
|---|---|---|---|---|
| Heart | NBP2-30178 | 2 | Myocytes | C |
| | FDA-Standard | 2 | Myocytes | C |
| Lymph node | NBP2-30182 | 2 | Dendritic reticulum of | C |
| | | | interdigitating reticulum cells | |
| Placenta | NBP2-30184 | 3 | Trophoblast | C + M |
| | FDA-Standard | 3 | Trophoblast | C + M |
| Skin | NBP2-30187 | 2 | Basal cells of epidermis | C |
| | FDA-Standard | 1 | Basal cells of epidermis | C |
| Tonsil | NBP2-30195 | 3 | Dendritic reticulum cells or | M |
| | | | interdigitating reticulum cells | |

TABLE 5
GPNMB Clinical trials

| Trial | Cancer | Phase | Patient # | Age | Adverse Effects | PMID |
|---|---|---|---|---|---|---|
| NCT00412828 | Melanoma | I/II | 117 | >18 | Generally Well Tolerated | 25267741 |
| NCT02302339 | Melanoma | II | 62 | >18 | Acceptable Safety Profile | 30690710 |
| NCT02363283 | Uveal Melanoma | II | 37 | >18 | Well Tolerated | 32823698 |
| NCT00704158 | Breast | I/II | 42 | >18 | Generally Well Tolerated | 25267761 |
| NCT01156753 | Breast Cancer | II | 120 | >18 | Well Tolerated | 25847941 |
| NCT01997333 | Breast Cancer | II | 213 | >18 | Well Tolerated | 34016993 |
| NCT02487979 | Osteosacroma | II | 22 | 12-49 | Well Tolerated | 31586757 |
| NCT02713828 | Lung Cancer | I | 13 | >18 | Well tolerated | 34590018 |

### Example 4—Development of a CAR Targeting GPNMB

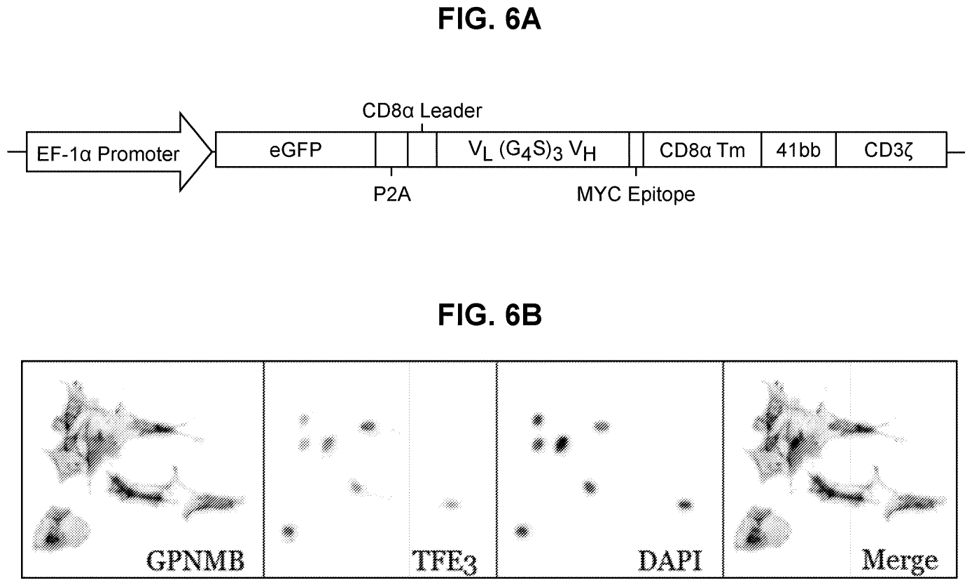

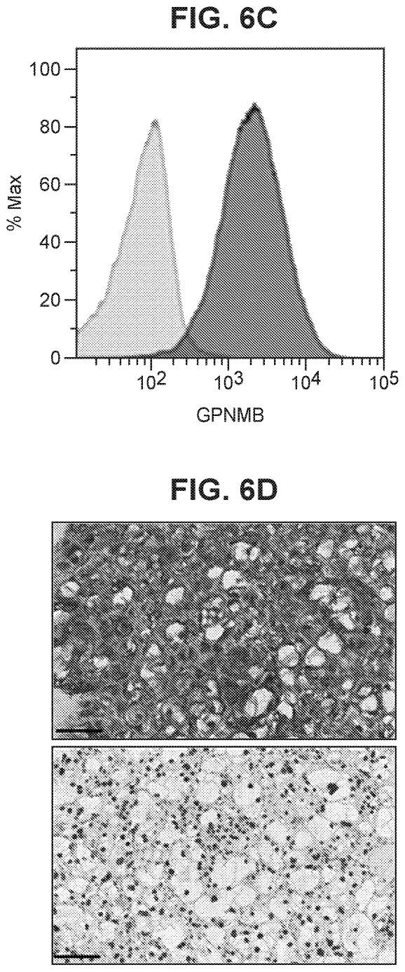

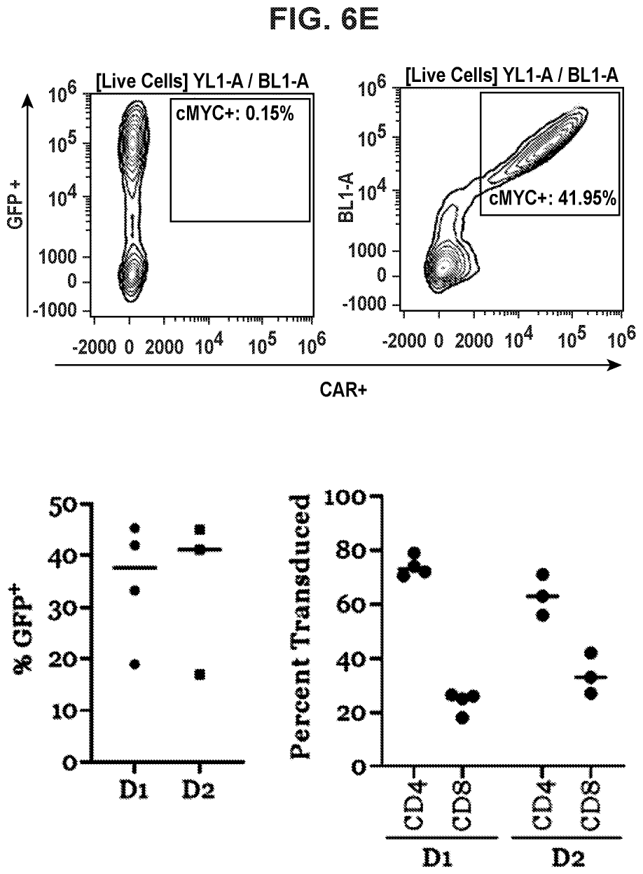

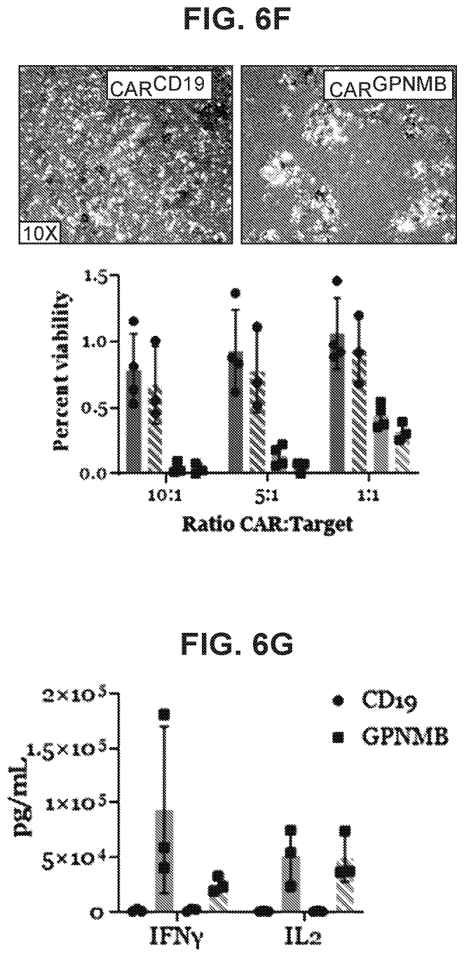

Given the highly elevated nature of GPNMB in ASPS, and the acceptable safety profile demonstrated in the TMAs and CDX-011 trials, second generation 41bbζ CAR T-cells (CAR Ts) targeting GPNMB were developed. A MYC-epitope tag was included between the scFv and the post-linker for measuring CAR expression, and a P2A sequence separating it from an EGFP protein was included to measure transduction efficiency (FIG. 6a). The domains and amino acid sequences thereof of the CARs employed in this example are provided in the table below (Table 6).

TABLE 6
CAR domains and amino acid sequences

| CAR Portion | Sequence |
|---|---|
| CD8a Leader | MALPVTALLL PLALLLHAAR P (SEQ ID NO: 63) |
| GPNMB | EIVMTQSPAT LSVSPGERAT LSCRASQSVD NNLVWYQQKP |
| Light Chain (G1) | GQAPRLLIYG ASTRATGIPA RFSGSGSGTE FTLTISSLQS |
| | EDFAVYYCQQ YNNWPPWTFG QGTKVEIKR (SEQ ID NO: 1) |
| GPNMB | DIVMTQSPLS LPVTPGEPAS ISCRSSQSLL HSNGYNYLDW |
| Light Chain (G2) | YLQKPGQSPQ LLIYLGSNRA SGVPDRFSGS GSGTDFTLKI |
| | SRVEAEDVGV YYCMQGLQTP ITFGQGTRLE IKR (SEQ ID NO: 15) |
| GPNMB | DIQLTQSPSS LSASVGDRVT ITCRASQGIR NDLGWYQQKP |
| Light Chain (G3) | GKAPKRLIYA ASSLQNGVPS RFSGSGSGTE FTLTISSLQP |
| | EDFATYYCLQ HNTYPAFCQG TKVEIKR (SEQ ID NO: 29) |
| GPNMB | LDVVMTQSPL SLPVTPGEPA SISCRSSQSL LHSNGYNYLD |
| Light Chain (G4) | WYLQKPGQSP QLLIYLGSNR ASGVPDRFSG SGSGTDFTLK |
| | ISRVEAEDVG VYYCMQALQT HPTFGQGTKV EIKR (SEQ ID NO: 43) |
| scFv Linker | GGGGSGGGGS GGGGS (SEQ ID NO: 54) |
| GPNMB | QVQLQESGPG LVKPSQTLSL TCTVSGGSIS SFNYYWSWIR |
| Heavy Chain (G1) | HHPGKGLEWI GYIYYSGSTY SNPSLKSRVT ISVDTSKNQF |
| | SLTLSSVTAA DTAVYYCARG YNWNYFDYWG QGTLVTVSSA (SEQ ID |
| | NO: 6) |
| GPNMB | QLVESGGGVV QPGRSLRLSC AASGFAFSSY GMHWVRQAPG |
| Heavy Chain (G2) | KGLEWVAVIS YDGNNKYYAD SVKGRFTISR DNSKNTLYLQ |
| | MNSLRAEDTA VYYCARDLVV RGIRGYYYYF GMDVWGQGTT VTVSSA |
| | (SEQ ID NO: 20) |
| GPNMB | QVQLEQSGPG LVKPSQNLSL TCTVSGGSIS SGGYFWSWIR |
| Heavy Chain (G3) | QHPGKGLEWI GYIYYSGNTY YNPSLKSRVT ISVDTSKNQF |
| | SLKLSSVTAA DTAVYYCARD YYYDTSGFSY RYDWYYGMDV |
| | WGQGTTVTVS SA (SEQ ID NO: 34) |
| GPNMB | QVQLVQSGAE VKKPGSSVKV SCKASGGTFS SYAISWVRQA |
| Heavy Chain (G4) | PGQGLEWMGG IIPIFGTANY AQKFQGRVTI TADESTSTAY |
| | MELSSLRSED TAVYYCARGP NTWGQGTLVT V (SEQ ID NO: 45) |
| MYC Tag | EQKLISEEDL (SEQ ID NO: 57) |
| Post-Linker | AAATTT (SEQ ID NO: 58) |
| CD8a Hinge | PAPRPPTPAP TIASQPLSLR PEACRPAAGG AVHTRGLDFA CD (SEQ |
| | ID NO: 59) |
| CD8a | IYIWAPLAGT CGVLLLSLVI TLYC (SEQ ID NO: 60) |
| Transmembrane | |
| 41bb | KRGRKKLLYI FKQPFMRPVQ TTQEEDGCSC RFPEEEEGGC EL (SEQ |
| | ID NO: 61) |
| CD3ζ | RVKFSRSADA PAYQQGQNQL YNELNLGRRE EYDVLDKRRG |
| | RDPEMGGKPR RKNPQEGLYN ELQKDKMAEA YSEIGMKGER |
| | RRGKGHDGLY QGLSTATKDT YDALHMQALP PR (SEQ ID NO: 62) |

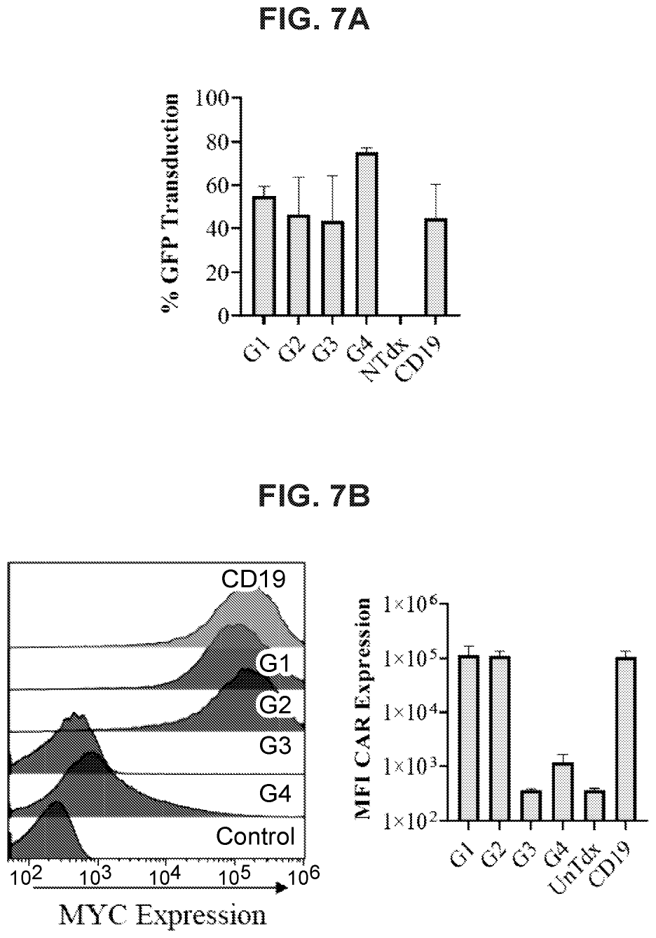

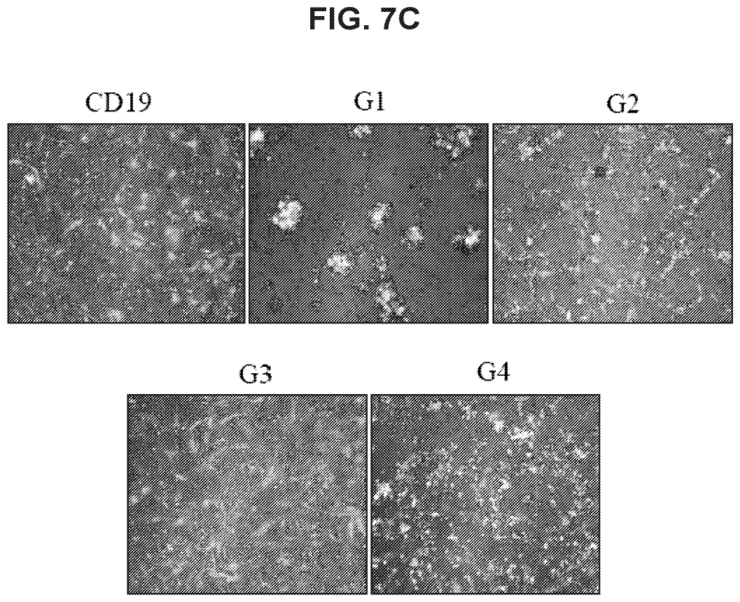

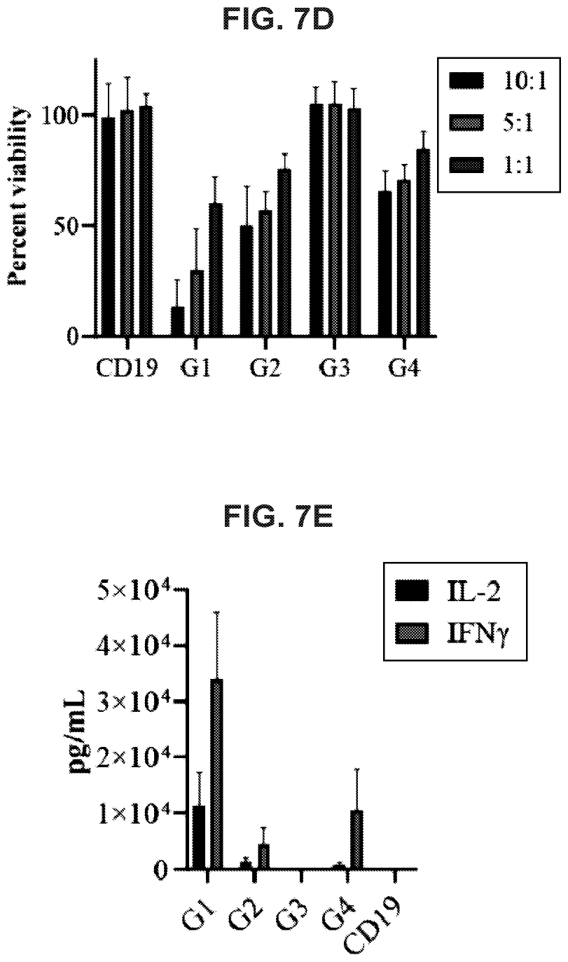

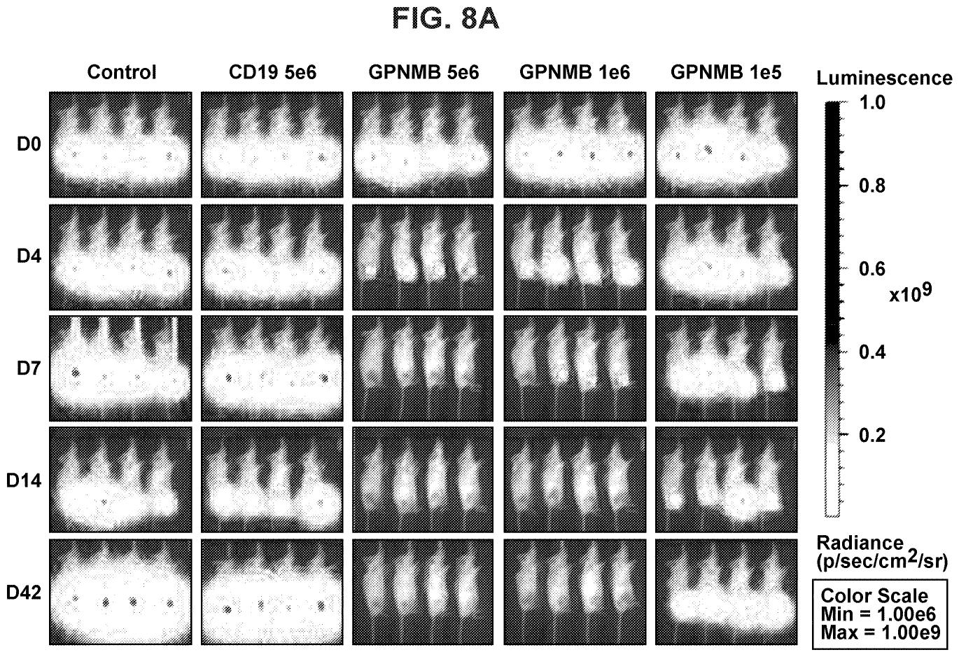

To test the effectiveness of the GPNMB CAR, an ASPS patient-derived cell line cultured from a lung metastases was employed (FIG. 1d). This model contains a type II ASPSCR1-TFE3 fusion, with nuclear localization of TFE3 and cell-surface GPNMB expression in vitro (FIG. 6b-c). Further, this model retained the histological characteristics of ASPS when grown orthotopically as a primary tumor in NSG mice with concordantly high-levels of GPNMB (FIG. 6d). Four separate binders (G1-G4) were tested on the ASPS PDX-derived cell lines. Unpredictably, and despite having similar transduction efficiency (FIG. 7a), only binders G1 and G2 demonstrated robust CAR surface expression (FIG. 7b). Testing these CARs head-head in in vitro co-culture experiments with ASPS PDX-derived cell line demonstrated that G1 had superior CAR-T activation/expansion, cytotoxicity and cytokine secretion capabilities (FIG. 7c-e). This G1 binder was used for the remainder of the preclinical studies and henceforth referred to as the GPNMB CAR. GPNMB CAR activity was elaborated on in two sets of healthy donors and good transduction efficiency of the GPNMB CAR at 5 MOI in both CD4 and CD8 T cell populations was demonstrated (FIG. 6e). When co-cultured with ASPS^{mCherry/FLUC} cells, the GPNMB CAR T-cells effectively killed (FIG. 6f) and secreted activation-associated cytokines (FIG. 6e).

### Example 5—In Vivo Efficacy of a GPNMB CAR T Product in a Model of ASPS

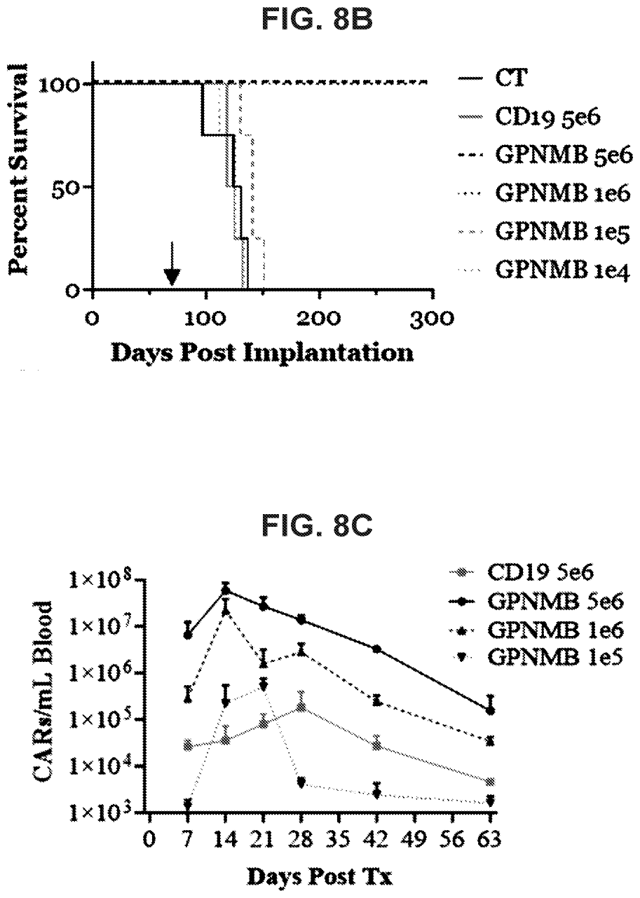

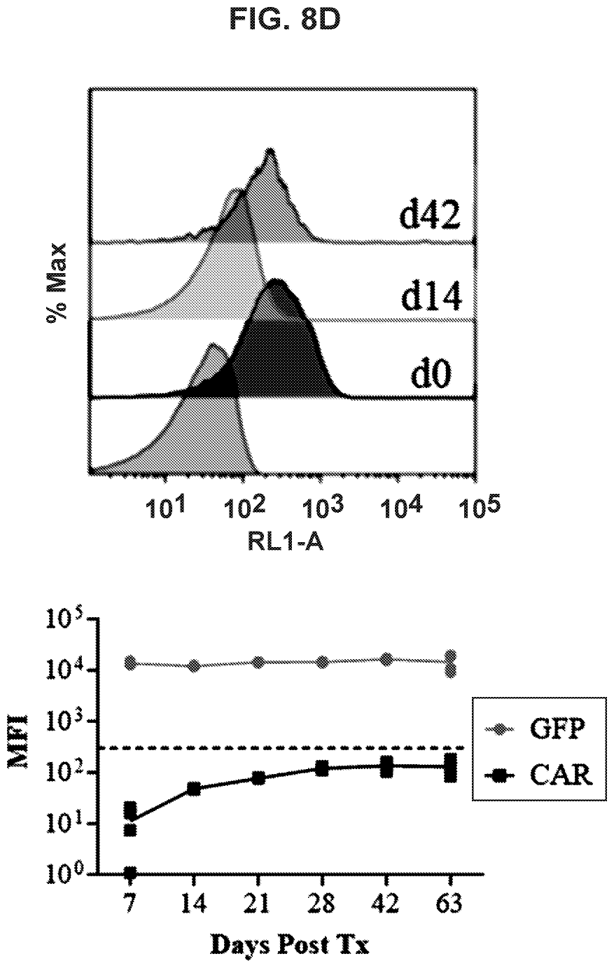

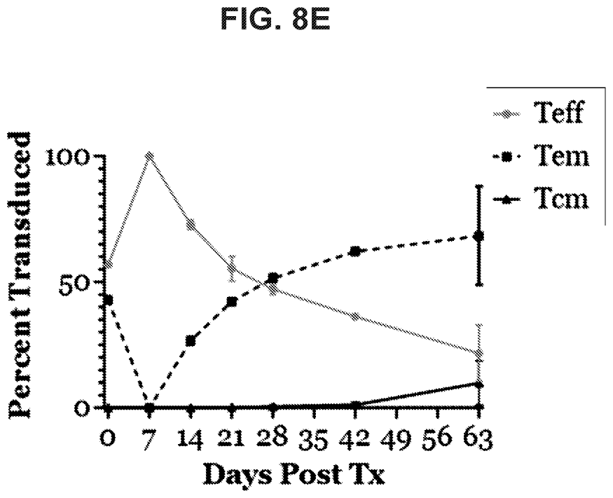

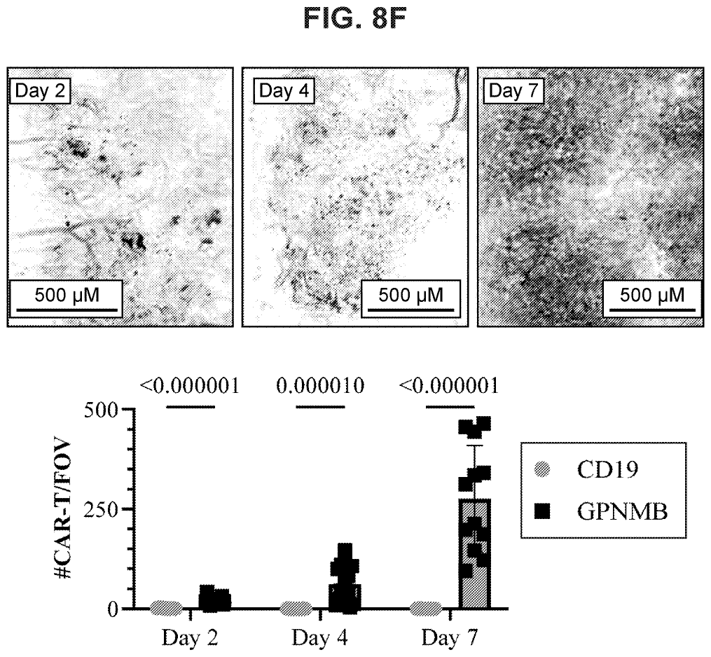

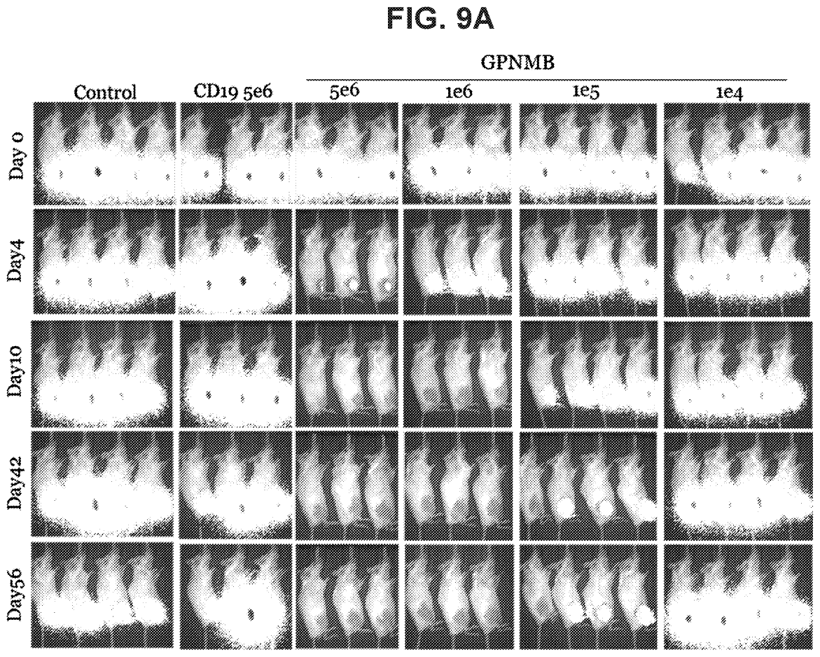

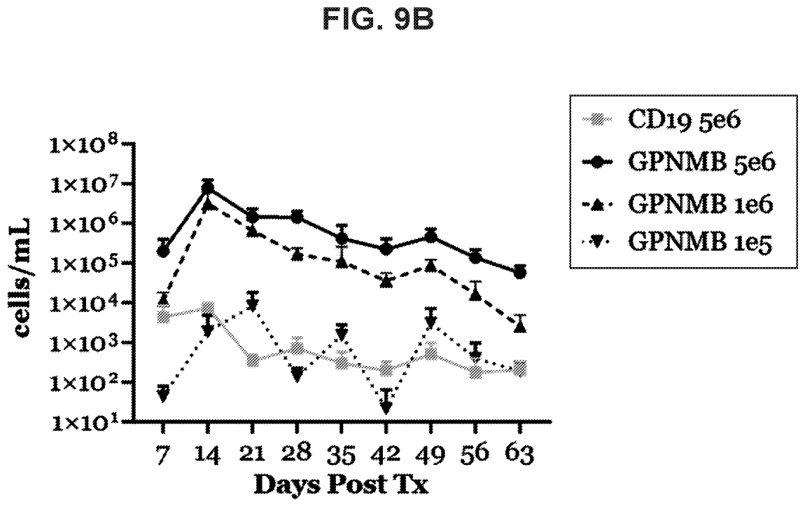

Having demonstrated the function of the GPNMB CAR in vitro, its efficacy was tested in an orthotopic model of ASPS primary disease. The ASPS patient-derived cell line was implanted intramuscularly into NSG-MHC^{KO} (NSG) mice and treated with various doses of GPNMB CAR T-cells or CD19 CAR T-cells. An immediate tumor reduction was observed starting at 4 days-post treatment with 5e6 and 1e6 GPNMB CAR T-cells, with complete elimination of tumor between 7- and 14-days post-treatment (FIG. 8a). Mice with cleared tumors did not relapse out to 300 days post treatment (FIG. 8b). Efficacy at the 1e5 dose was also observed, resulting in a maximal 68±25% reduction in tumor size as measured by bioluminescence at day 21. These mice eventually relapsed with a mild overall survival advantage (FIG. 8b) but retained GPNMB expression. 1e4 GPNMB CAR T-cells per mouse resulted in no therapeutic benefit. Tumor efficacy coincided with CAR T-cell expansion in the periphery (FIG. 8c), with an average of 5.92e7±2.60e7 cells/mL at peak expansion at day 14 in the 5e6 CAR T-cell dose, corresponding to a 1668±733-fold expansion of GPNMB CAR T-cells over the non-targeting CD19 CAR T-cells. Significant expansion was also observed in the 1e6 and 1e5 dose, albeit peak expansion of the 1e5 CAR T-cells was observed at day 21. GPNMB CAR T-cell efficacy and expansion was repeated using separate healthy donor T cells with very similar results (FIG. 9). Also observed was a dramatic, albeit transient, loss of CAR expression on peripherally circulating CAR T-cells (FIG. 8d). This was attributed to CAR internalization as a result of target engagement, as the MFI of EGFP transduction marker remained unchanged. However, 28 days following treatment CAR levels returned to near original levels and remained stable. Peripherally circulating CAR T-cells were initially polarized to an effector phenotype (CD45RO⁻CD62L⁻), with the ratio of effector memory (CD45RO⁺CD62L⁻) to effector cells increasing until near pre-injection between days 28 and 42 (FIG. 8e). The emergence of memory CAR T-cells (CD45RO⁺CD62L⁺) started at day 42 and further increased at day 63. Finally, to measure and observe the expansion of the GPNMB CAR T-cells within tumors, intravital microscopy was employed (FIG. 8f). CAR Ts were seen to infiltrate to the tumor as early as day 2, with dramatic expansion seen through subsequent days. Four days post-treatment, an obvious inverse correlation was observed between areas of tumor and areas of expanding CAR T-cells. By day 7, any significant areas of tumor within any fields of microscopic view could not be found and all areas were dominated by extravascular CAR T-cells. Together, these data demonstrate that the GPNMB CAR T product is highly functional and effective systemic therapy in this proof-of-concept tumor model (primary ASPS tumors).

### Example 6—Eradication of CNS Metastasis by Systemic Administration of GPNMB CAR T-Cells

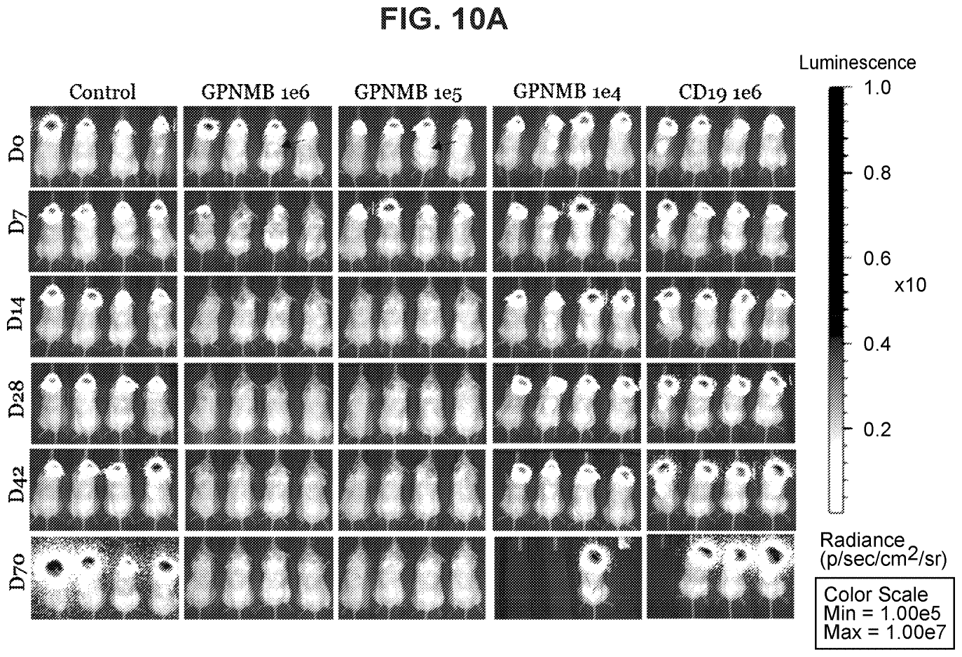

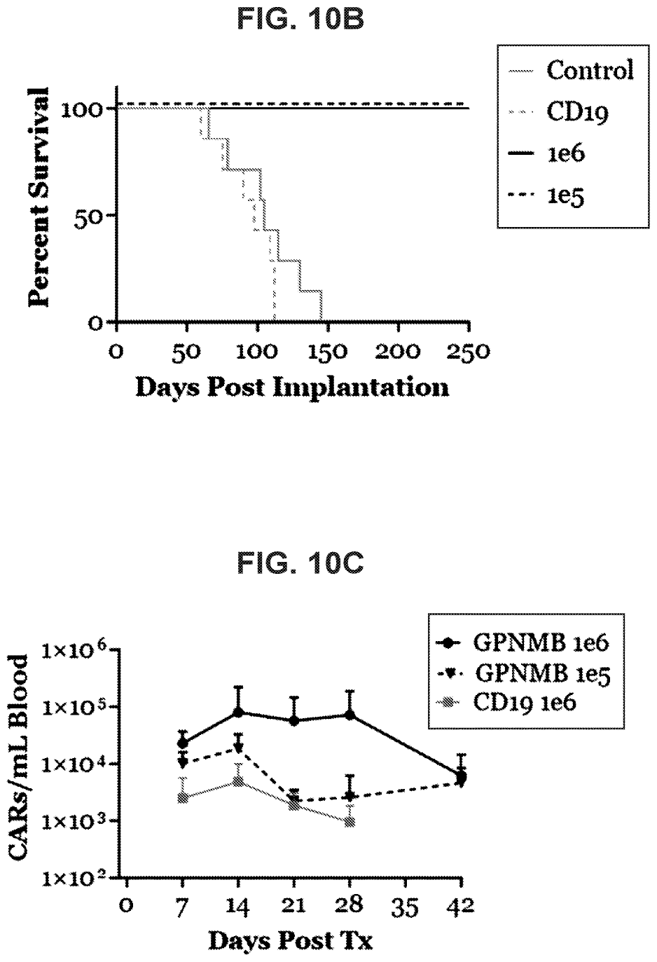

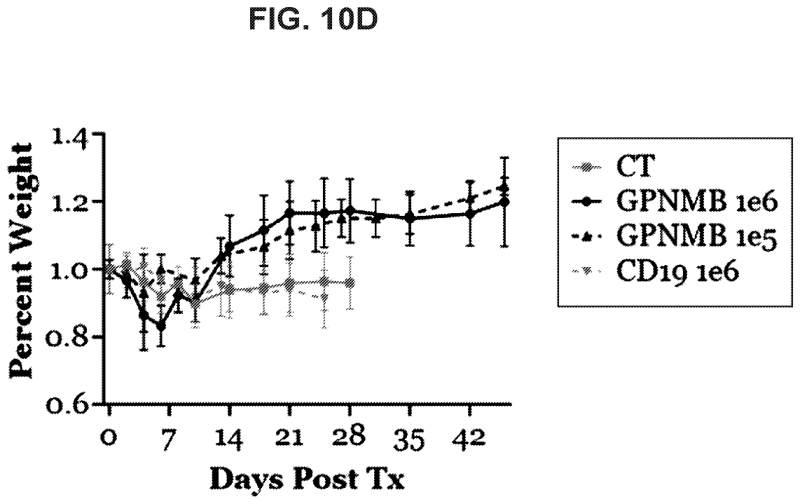

ASPS has the highest incidence of brain metastasis of all sarcomas (11-19%), which can be a significant cause of morbidity and mortality in patients. To test whether GPNMB CAR T-cells could be systemically administered to target ASPS CNS metastasis, the ASPS patient-derived cell line was implanted intracerebrally. Doses of 1e6 and 1e5 GPNMB CAR T-cells were surprisingly found to completely eradicate CNS metastasis by 7-14 days post treatment, resulting in a durable response with no tumor relapse (FIG. 10a-b). Further, in some instances, formation of spinal metastasis occurred in several animals by the time of treatment initiation (FIG. 10a, white arrows). Animals with bioluminescent signal in the spine that were treated with systemic GPNMB CAR T-cells completely resolved these lesions. Tumor regression was accompanied by CAR T-cell expansion in the periphery (FIG. 10c), albeit this was to a lesser extent than seen with the primary tumor model (8.00e4±1.4e5 vs. 2.23e7±1.65e7 CARs/mL at 1e6 CAR T-cells per mouse in intracranial vs. intramuscular, respectively), reaching a maximal of 16.5-fold increase over the non-targeting CD19 CAR T-cells at 14 days post treatment.

Interestingly, systemic administration of the GPNMB CAR T-cells to intracranial ASPS tumors marked the first observation of any treatment-related toxicity to this therapy. Mice treated with 1e6 GPNMB CAR T-cells, but not the 1e5 GPNMB CAR T-cells, demonstrated an immediate and rapid weight loss that started to resolve 7 days after treatment (FIG. 10d). Neuropathological assessment of the brains of treated mice demonstrated no residual tumor and an absence of any obvious neurological damage. This observation paired with the fact that toxicity was only observed in animals with a high dose of CAR T-cells, led us to postulate that this toxicity was due to the rapid, simultaneous expansion of CAR T-cells in the intracranial space leading to transient intracranial oedema.

### Example 7—Clinical Translation of GPNMB CAR

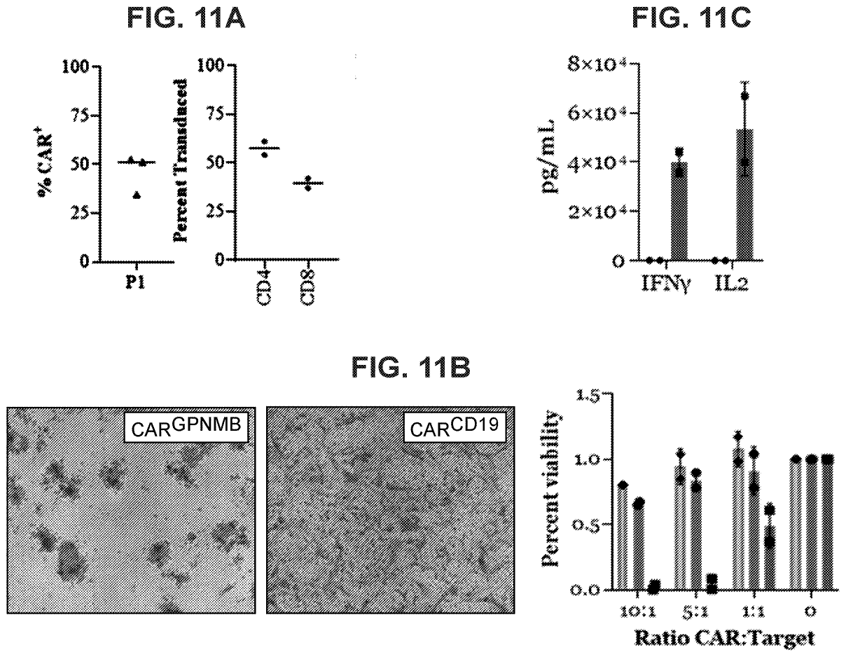

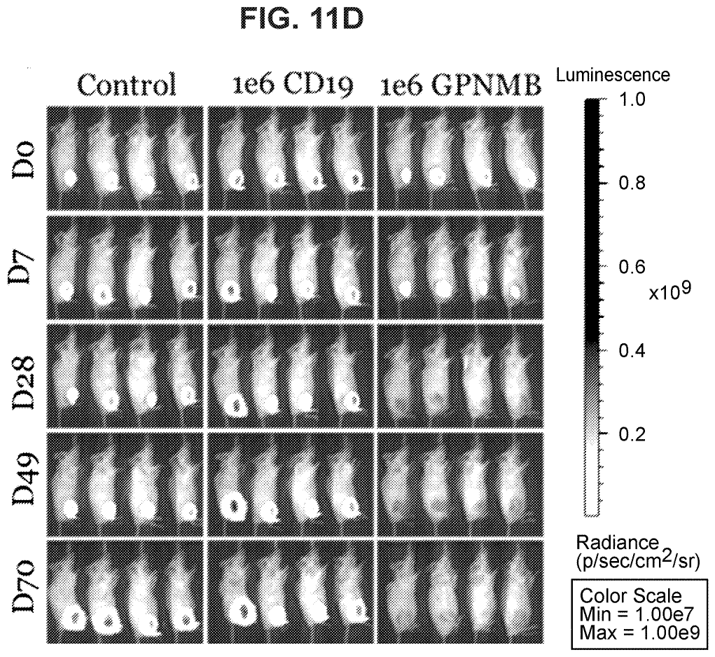

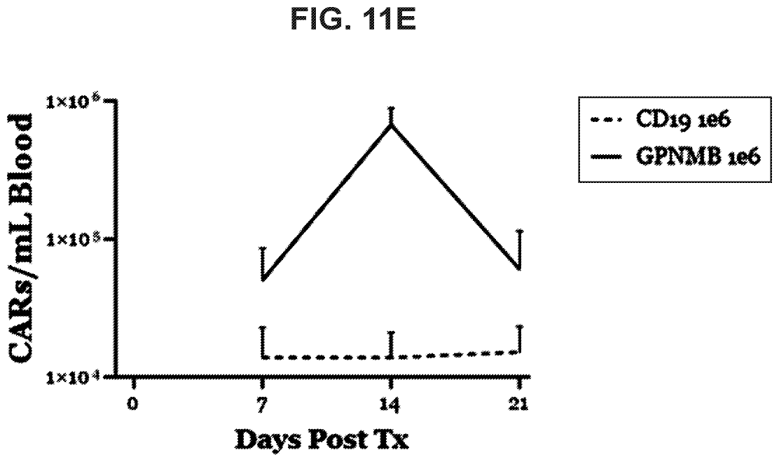

Given the efficacy observed in the in vivo model of ASPS and the fact that metastatic ASPS is considered ‘incurable’ with limited systemic therapy options, a clinical translation effort of the GPNMB CAR for ASPS was initiated. To begin, ASPS patient T cells were tested for GPNMB transduction efficiency and in vitro activity on the matched patient-derived cell line. In vitro analysis demonstrated comparable transduction efficiency to healthy donor cells (FIG. 11a), and equally potent cytotoxic activity and cytokine secretion (FIG. 11b). Further, treating the ASPS mouse model resulted in complete regression of the primary tumor with 1e6 CAR T-cells per mouse, as was observed using healthy donor-derived GPNMB CAR T-cells (FIG. 11c).

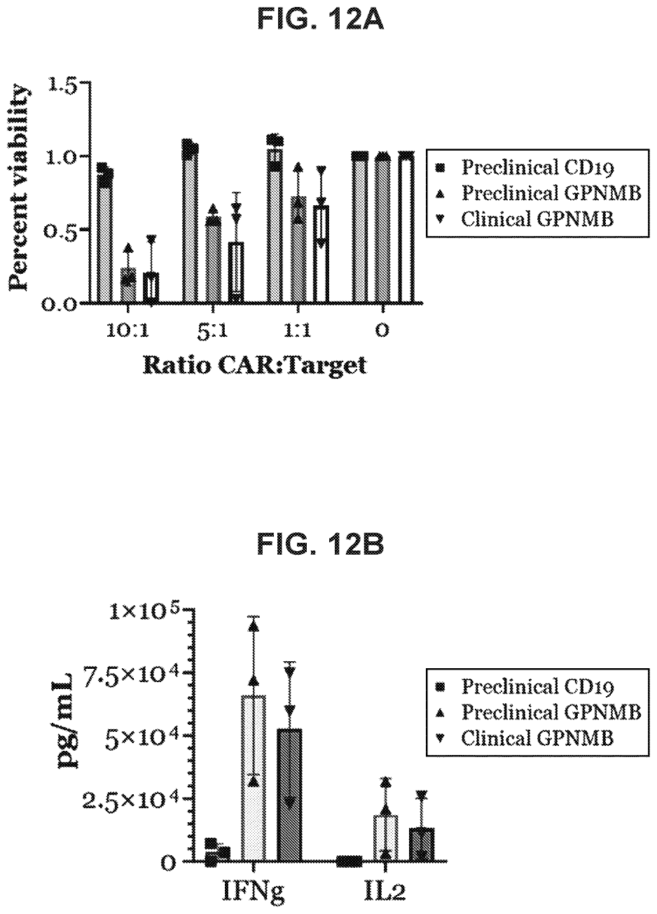

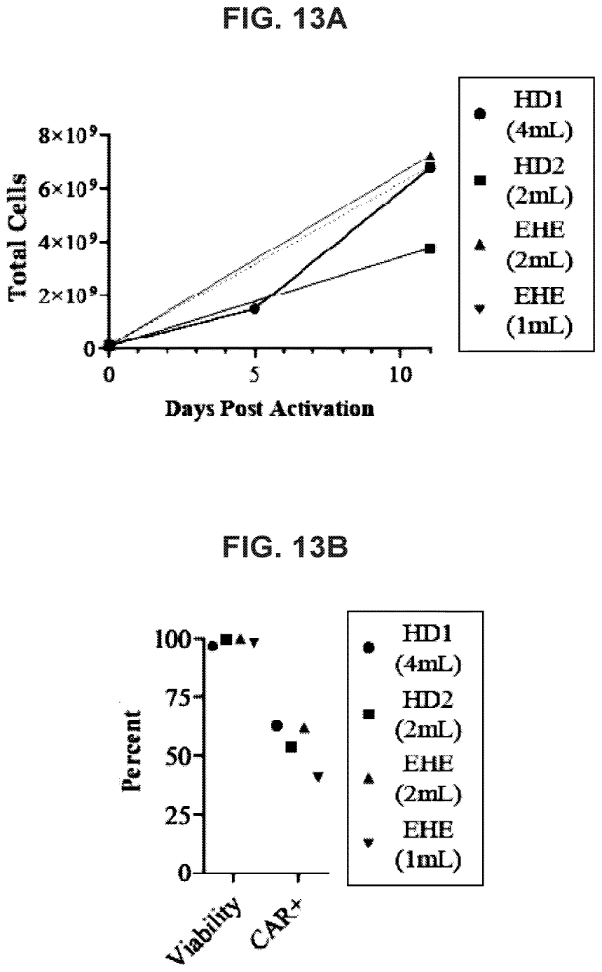

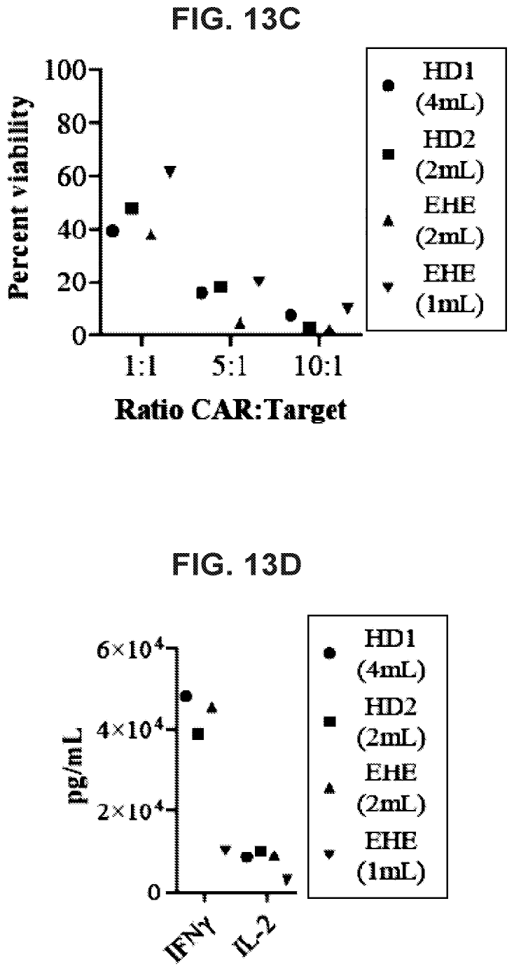

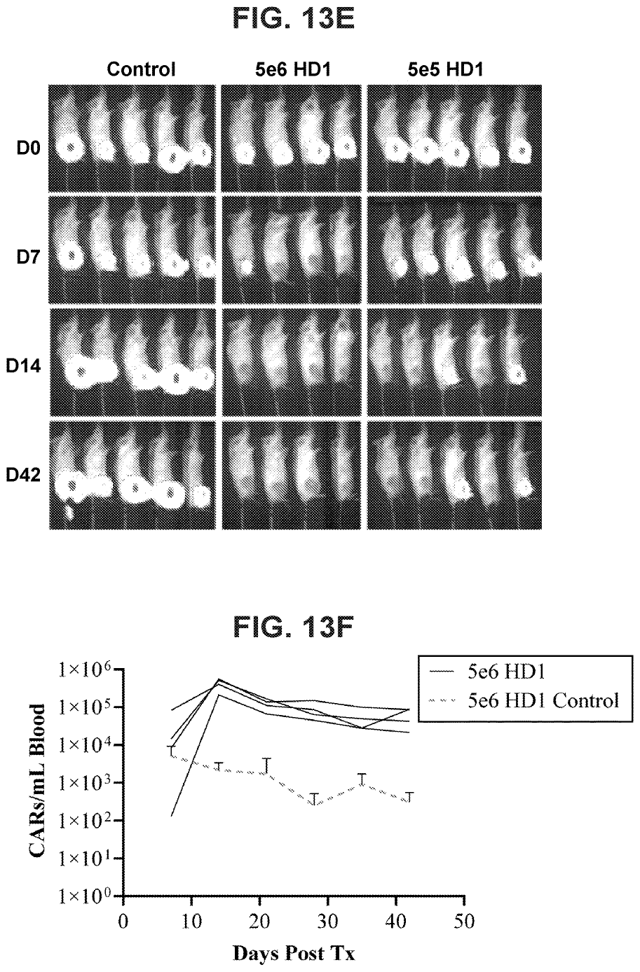

Constructed next was a clinical GPNMB CAR vector, placing the identical GPNMB targeting sequence into the CD19-41BBζ-CLIC backbone currently in clinical trial in Canada (NCT03765177) and the removal of the EGFP protein. Direct head-to-head comparison of the preclinical and clinical CAR constructs demonstrated that the CAR behaved similarly when transduced into patient T cells (FIG. 12). Subsequent production of the clinical-grade lentiviral vector under good manufacturing practice (GMP) produced 86 mL of 1.0e7 IU/mL titered virus. Four mock clinical runs on the CliniMACs© prodigy have been performed using different volumes of clinical-grade lentivirus in both healthy donor (HD) and cancer patient (EHE) apheresis material. All runs on the CliniMACs© demonstrated robust T cell expansion of viable CAR product (FIG. 13a-b). Potency of these products was assessed on the ASPS PDX-derived cell line and measured by a cytotoxicity assay and cytokine secretion (FIG. 13c-d). All products passed quality assurance specifications (Table 7). Product from the HD1 run were tested in the orthotopic model of ASPS primary disease, and demonstrated complete tumor clearance in all mice at a dose of 5e6 CARs/mouse, and in 3 of 5 mice at 5e5 CARs/mouse (FIG. 13e). Significant CAR T expansion and persistence was also observed with the clinical-grade product in the ASPS model when compared to mice bearing a GPNMB-negative tumor (FIG. 14c, 786-O, a ccRCC line) and treated with the same dose (FIG. 13f).

TABLE 7
Quality assurance specifications of products

| ANALYTICAL METHOD | SPECIFICATION | HD1 (4 mL) | HD2 (2 mL) | EHE (2 mL) | EHE (1 mL) |
|---|---|---|---|---|---|
| Harvest Product Purity (% CD3 of total cells) | ≥90% | 94.94 | 97.45 | 92.81 | 100.00 |
| Harvest Product Viability (% Live) | ≥70% | 96.70 | 99.80 | 100.00 | 98.00 |
| Harvest Product CAR Expression (% Total cells) | To Report | 63.02 | 53.57 | 62.14 | 40.82 |
| Drug Product Appearance | Trans., NP | Trans., NP | Trans., NP | Trans., NP | Trans., NP |
| Drug Product pH | 7-7.5 | 7-7.5 | 7-7.5 | 7-7.5 | 7-7.5 |
| Drug Product Sterility - Anaerobic | No growth | No growth | No growth | No growth | No growth |
| Drug Product Sterility - Aerobic | No growth | No growth | No growth | No growth | No growth |
| Drug Product Sterility - Fungal | No growth | No growth | No growth | No growth | No growth |
| Drug Product Mycoplasma | Not detected | Not detected | Not detected | Not detected | Not detected |
| Drug Product Endotoxin (EU/mL) | Run/formulation-dependent | <0.050 | <0.050 | <0.500 | <0.500 |
| Drug Product Vector Copy # (/CAR+ T cell) | ≤5 copies | 1.55 | 1.01 | 1.35 | 1.40 |

### Example 8—Indication Expansion for GCAR1

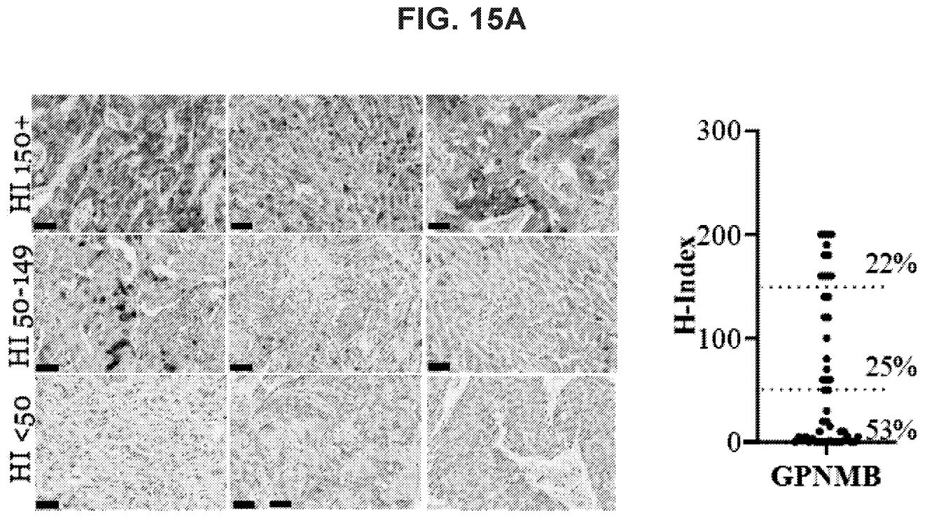

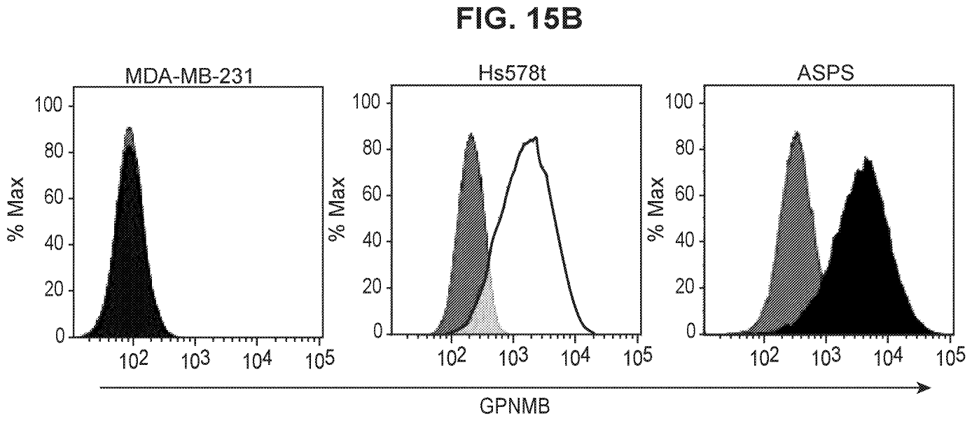

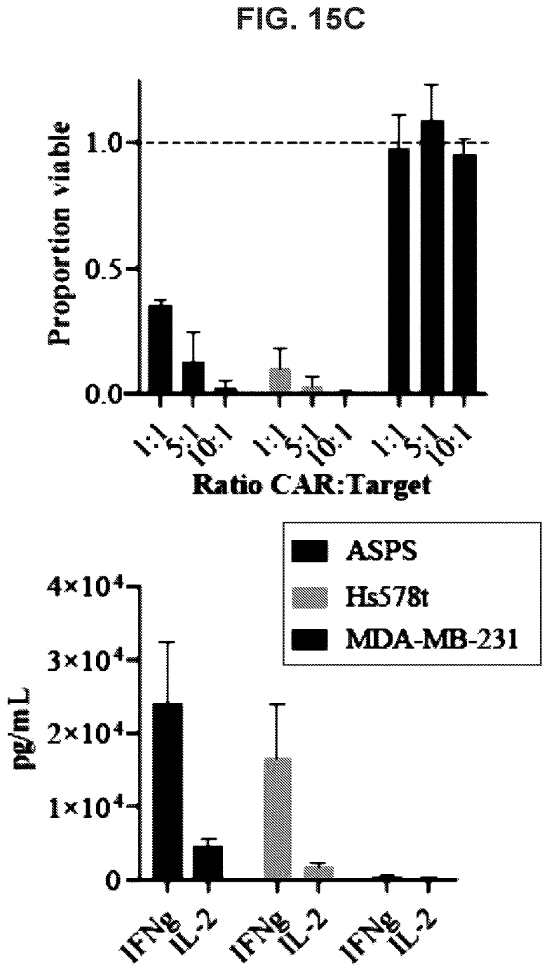

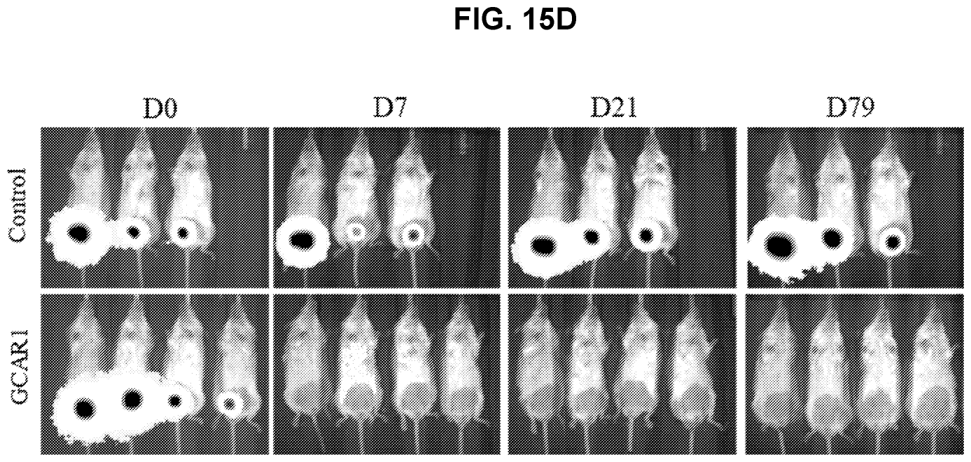

GPNMB is known to be regulated by MiTF family members in the steady state and has previously been demonstrated as a transcriptional target of Xp11 translocation positive (t)RCC fusion proteins resulting in high and homogeneous upregulation of GPNMB (Tanaka et al. (2017) Cancer Res 77:897-907; Baba et al. (2019) Mol Cancer Res 17:1613-1626) and TSC/mTOR altered RCC (Salles et al. (2022) J Pathol. 257(2): 158-171). A panel of TSC/mTOR altered RCC and translocation positive tRCC patients were assessed for GPNMB expression, and high and homogenous GPNMB expression was confirmed in most instances of these RCC subtypes (FIG. 14a-b). Next, a panel of fusion positive tRCC cell lines and fusion negative RCC cell lines were tested, finding only tRCC lines demonstrated cell surface GPNMB expression (FIG. 14c). FU-UR1, UOK-124 and UOK-146 (tRCCs) and 786-O (RCC) were subcutaneously implanted into mice and treated with 5e6 CARs/mouse of clinical grade HD1 GPNMB CAR product. tRCC models displayed a robust treatment response to the GPNMB CAR, accompanied by significant product expansion in the blood. These responses were not observed in the RCC (GPNMB−) model (FIG. 14d-e). A tissue-microarray of 49 TNBC was also evaluated, demonstrating variable GPNMB expression (FIG. 15a) as others have demonstrated. TNBC cell lines MDA-MB-231 and Hs578T were evaluated for cell surface expression of GPNMB compared to positive control ASPS (FIG. 15b), demonstrating GPNMB expression in the Hs578T line. The TNBC lines were tested head-head for cytotoxicity and cytokine secretion when treated in co-culture experiment with HD1 clinical-grade GPNMB CAR (FIG. 15c), demonstrating GPNMB CAR-specific cytotoxic and cytokine responses to the GPNMB+TNBC. Luciferase-expressing Hs578T cells were implanted in the mammary fat pad of NSG-KO mice and treated with 1e6 GPNMB CAR-T cells from HD1 demonstrating robust and durable response in this model (FIG. 15d).

### Materials and Methods

### Differential Expression in ASPS Samples

A pre-existing, microarray-based gene expression profile of ASPS (GSE13433, 14 ASPS samples and two universal RNAs) was downloaded from GEO. First, log-transformed and normalized expression values were mapped at the probe level to the official gene symbols using the R package biomaRt, and the level of gene expression was consolidated by taking the average across probes mapping to the same gene symbol, which yielded 17,545 genes in total. The technical duplicates for each ASPS tumor, n=7, were then averaged before assessing differential expression (DE). 5,725 likely cell surface proteins (genes) were selected using an integrated database of the surfaceome, where four databases (Cell Surface Protein Atlas, COMPARTMENTS, Human Protein Atlas, SURFY) were integrated to prioritize them. Eleven DE genes identified with SurfaceGenie, score>2 were considered as the final candidates. The DE genes were obtained from Welch's t-test between the ASPS tumor and universal RNA groups and false discovery rate (FDR) corrections were applied. Genes were considered DE if FDR<0.01 and log 2-fold-change>0.5 (i.e., ASPS-specific gene). Lastly, two evaluation datasets (GSE32569 and GSE49327) were downloaded from GEO where there were only ASPS tumor profiles (i.e., pre- or post-treatment, primary or metastasis), and they were processed the same way described above to confirm the level of expression in top candidates.

### GPNMB Expression Across Tissues

Two proteomics databases, Human Protein Map and Wang et al., were localized to check the level of GPNMB expression across tissues in non-malignant samples. In addition, CD276 and HER2 were included as established CAR targets and 14 important housekeeping genes (https://www.genomics-online.com/resources/16/5049/housekeeping-genes/): ACTB, B2M, GAPDH, GUSB, HMBS, HPRT1, PGK1, PPIA, RPL13A, RPLP0, SDHA, TBP, TFRC, and YWHAZ Since the range of the mass spectrometry (MS) expression varies and can be extreme, the MS values were capped at the 90th percentile across 17 proteins within the dataset. Then, the MS values were scaled from [minimum, maximum] expression to [0, 1].

### CAR Generation, Preclinical Vector Manufacturing and In Vitro Testing

Lentiviral plasmids containing CAR constructs were generated by standard molecular cloning methods. The preclinical GPNMB CAR construct was assembled from a human scFv sequence recognizing GPNMB, a MYC epitope tag, a CD8α hinge and transmembrane domain, and 4-11BB and CD3ζ intracellular signaling domains. The scFv employed in this examples comprises the V_{L} and V_{H} of the antibody present in the antibody-drug conjugate (ADC), glembatumumab vedotin (CDX-011-MMAE). See, e.g., WO2006071441A2.

This construct was cloned into a pULTRA-EGFP vector (Addgene #24129) downstream of the EGFP and separated by a P2A site. The UbC promoter was changed for a full length EF1α promoter. An identical CD19(FMC63)-targeting construct was also made in this vector.

Preclinical lentivirus particles were packaged in LentiX 293T (Takara) cells using packaging plasmid pCMV-dR8.91, envelope plasmid pMD2.G, and CAR construct (5:1:5 ratio). Supernatants containing lentivirus particles were collected 48 hours after transfection and concentrated by ultracentrifugation. Viral titer in transduction units per milliliter was determined by flow cytometry analysis of transduced Lenti-X 293 cells.

Human PBMCs were isolated from healthy donor or patient blood by Ficol-paque density centrifugation method. CD3 positive T cells were sorted from PBMCs by CD3 isolation kit from Miltenyi Biotec. Isolated CD3 positive cells were cultured in TexMACs T-cell expansion medium supplemented with IL7 and IL15 (BioLegend; 10 ng/mL) and activated with CD3/28 Transact beads (Miltenyi Biotec). 24 hrs after activation, CAR-containing lentiviruses were used to transduce T cells at a multiplicity of infection of 5. Cells were then expanded for an additional 7 to 9 days.

The ASPS patient-derived cell line was transduced with a lentiviral construct containing mCherry and firefly luciferase. ASPS^{mC/herryFLUC} was plated in 96-well plate and treated with indicated ratios of CAR T-cells for 24 hours. Cytotoxic activity was quantified by adding luciferin (GoldBio) to a final concentration of 150 μg/mL and measured on a SpectraMax i3. IL2 and IFNγ were measured by ELISA kits (BioLegend) and read on the SpectraMax i3.

### In Vivo CAR T Testing

The ASPS-patient-derived cell line was obtained from a clinical biopsy collected from a 17-year old female with ASPS undergoing surgery for pulmonary metastases. Fresh tumor tissue was implanted into the flank of SCID mice and was established as a patient-derived xenograft (PDX). Established PDX tissue was removed, a single cell suspension by gently trituration, and plated in complete OptiMEM media to establish a patient-derived ASPS cell line.

Eight- to ten-week-old female NOD.Cg-Prkdc^{scid}H2-K1^{tm1Bpe}H2-Ab1^{emMvw}H2-D1^{tm1Bpe}II2rg^{tm1Wijl}/SzJ (Jackson laboratory; strain #025216) were used in this study. For modelling primary disease, 1e6 ASPS^{mC/FLUC} were injected in the gastrocnemius. Intracranial metastasis was modelled by steriotactically implanting 1e6 ASPS^{mC/FLUC} into the right striatum of mice. 35- 42 days following implantation, mice were treated with indicated number of CAR T-cells intravenously and tumor burden was evaluated by bioluminescence imaging using the Xenogen system and processed with LivingImage Software. Weekly blood draws for measuring CAR T in peripheral blood was performed by saphenous bleeds in EDTA tubes. For other animals models, 5e6 cells from each of the RCC cell lines were injected subcutaneously in the same strain of mice in matrigel. CAR Treatment was initiated when tumors reached an average volume of 150-250 mm³. 5e6 Hs578T cells were implanted in the mammary fat pad of this strain and treated 47 days after implantation. Animals were monitored daily for general well-being until they reached humane or experimental endpoint. Animal experiments were approved by The University of Calgary Conjoint Health Research Ethics Board.

### Flow Cytometry

Single cells derived from cell culture were resuspended in PBS+2% FBS+0.25 mM EDTA (flow buffer). Alternatively, blood derived from saphenous bleeds was processed in ACK and resuspended in flow buffer. For GPNMB staining of ASPS patient-derived cell line, anti-GPNMB (R&D MAB25501) with anti-mouse AF647 (BioLegend) antibodies were used. For staining in vitro- and in vivo-derived T cells, antibodies from BioLegend were used. Samples were run on an ATTUNE flow cytometer and data analyzed with Kaluza software.

### Immunohistochemistry and Immunofluorescence

Tissue sections were deparaffinized with xylene and rehydrated through an ethanol gradient. Heat-induced epitope retrieval was performed using a microwave with slides in 1× antigen retrieval buffer (10 mM Citrate buffer pH 6.0+0.05% Tween 20) for 20 minutes at 95° C. Endogenous peroxidase activity was blocked by adding 1 drop of Peroxidase-Blocking Solution (Dako, S202386-2) to each slide and incubating for 15 minutes. Nonspecific binding was blocked with 200 μL of protein block (Agilent, X090930-2) with 0.2% Triton X-100 (Sigma, Oakville) and incubated for 20 minutes. Each section was incubated for 1 hour or overnight with goat polyclonal anti-human GPNMB antibody (R&D systems, AF2550, 1:250) or rabbit polyclonal anti-human TEF3 (Sigma-Cellmarque 354R-15 1:200). After washing each section with TBS, 1 drop of the appropriate polymer-HRP secondary antibody (Vector Laboratories or DAKO Envision) was applied, incubated for 30 minutes, washed with TBS, and detected using DAB reagent (DAKO, 1 drop DAB in 1 mL DAB substrate solution). Sections were counterstained with Hematoxylin, then dehydrated through an ethanol gradient, and sections were mounted with Entellan (Electron Microscopy Sciences). The stained slides scanned using Aperio Scanscope® XT (Aperio Inc.), slide scanner at 20× or 40× resolution and images were acquired, using Imagescope v12.2.2.5015 software.

ASPS cell lines were cultured in OPTI-MEM+10% FBS for 48 h. The sections were fixed with 4% PFA and permeabilized using 0.5% Triston X-100. Each section was incubated with goat polyclonal anti-human GPNMB antibody (R&D systems, AF2550, 20 ug/mL) and rabbit monoclonal anti-TFE3 (Sigma-Cellmarque, 354R-15, 1:25) ON at 4° C. The secondary antibody Alexa Fluor®647 Goat anti-mouse IgG (Biolegend, 405322) was added for GPNMB detection and DyLight™ 488 Donkey anti-rabbit IgG (Biolegend, 406404) was added for TFE3 detection. Both were added at 1 ug/mL for 30 minutes. Sections were stained with one drop of ProLong™ Gold antifade reagent with DAPI (Invitrogen, P36935). The stained slides were digitalized using ECHO Revolve (ECHO, RVL-100-M) at 40× resolution.

### Clinical Vector and CAR T Production

Clinical GPNMB plasmid was assembled with an identical GPNMB targeting sequence and was cloned into the CD19-41BBζ-CLIC backbone currently in clinical use (NCT03765177). Plasmid were manufactured using GMP-sourced reagents and strict quality control at the BC cancer agency. GMP manufacturing of lentiviral particles was done by the Ottawa Hospital Research Institute Biotherapeutics manufacturing Center. Mock-clinical runs were performed on the Miltenyi Biotec CliniMACS Prodigy® using the standard T cell Transduction (TCT) process protocol and the CliniMACS Prodigy® TS 520 tubing set and according to the manufacturers protocol and SOPs of the Alberta Cellular Therapy Program. Healthy donor apheresis material was procured from StemCell Technologies. Apheresis material from a patient with Epithelioid Hemangioendothelioma (EHE; HREBA.CC-22-0367) was also utilized. This EHE patient had a confirmed YAP1-EHE fusion, and is a candidate for GPNMB CAR T therapy.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1.-44. (canceled)

45. A chimeric antigen receptor (CAR) comprising:
an extracellular glycoprotein NMB (GPNMB)-binding domain;
a transmembrane domain; and
one or more intracellular signaling domains.

46. The CAR of claim 45, wherein the extracellular GPNMB-binding domain comprises a single chain antibody that specifically binds GPNMB.

47. The CAR of claim 46, wherein the single chain antibody is a single chain variable fragment (scFv).

48. The CAR of claim 46, wherein the single chain antibody comprises or competes for binding to GPNMB with an antibody comprising:
a variable light chain (V_{L}) polypeptide comprising
a V_{L} CDR1 comprising the amino acid sequence QSVDNN (SEQ ID NO:2),
a V_{L} CDR2 comprising the amino acid sequence GAS, and
a V_{L} CDR3 comprising the amino acid sequence QQYNNWPPWT (SEQ ID NO:3); and
a variable heavy chain (V_{H}) polypeptide comprising
a V_{H} CDR1 comprising the amino acid sequence GGSISSFNYY (SEQ ID NO:7),
a V_{H} CDR2 comprising the amino acid sequence IYYSGST (SEQ ID NO:8), and
a V_{H} CDR3 comprising the amino acid sequence ARGYNWNYFDY (SEQ ID NO:9);
a variable light chain (V_{L}) polypeptide comprising
a V_{L} CDR1 comprising the amino acid sequence QSLLHSNGYNY (SEQ ID NO:16),
a V_{L} CDR2 comprising the amino acid sequence LGS, and
a V_{L} CDR3 comprising the amino acid sequence MQGLQTPIT (SEQ ID NO:17); and
a variable heavy chain (V_{H}) polypeptide comprising
a V_{H} CDR1 comprising the amino acid sequence GFAFSSYG (SEQ ID NO:21),
a V_{H} CDR2 comprising the amino acid sequence ISYDGNNK (SEQ ID NO:22), and
a V_{H} CDR3 comprising the amino acid sequence ARDLVVRGIRGYYYYFGMDV (SEQ ID NO:23), or
a variable light chain (V_{L}) polypeptide comprising
a V_{L} CDR1 comprising the amino acid sequence QSLLHSNGYNY (SEQ ID NO:16),
a V_{L} CDR2 comprising the amino acid sequence LGS, and
a V_{L} CDR3 comprising the amino acid sequence MQALQTHPT (SEQ ID NO:44); and
a variable heavy chain (V_{H}) polypeptide comprising
a V_{H} CDR1 comprising the amino acid sequence GGTFSSYA (SEQ ID NO:46),
a V_{H} CDR2 comprising the amino acid sequence IIPIFGTA (SEQ ID NO:47), and
a V_{H} CDR3 comprising the amino acid sequence ARGPNT (SEQ ID NO:48),
wherein numbering of CDRs is according to IMGT.

49. The CAR of claim 48, wherein the single chain antibody comprises:
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:1, SEQ ID NO:15, or SEQ ID NO:43; or
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:6, SEQ ID NO:20, or SEQ ID NO:45.

50. A nucleic acid encoding the CAR of claim 45 or an expression construct comprising the nucleic acid operably linked to a promoter.

51. A cell comprising the expression construct of claim 50, wherein the cell expresses the CAR on its surface.

52. The cell of claim 51, wherein the cell is an immune cell or a stem cell.

53. The cell of claim 52, wherein the cell is a T cell.

54. A composition comprising a populations of cells as defined in claim 51.

55. A method of treating a condition associated with GPNMB expression and/or activity in a subject in need thereof, the method comprising administering an effective amount of the composition of claim 54 to the subject.

56. The method according to claim 55, wherein the condition associated with GPNMB expression and/or activity is cancer.

57. The method according to claim 56, wherein the cancer is characterized by GPNMB expressing (GPNMB+) cancer cells.

58. The method according to claim 56, wherein the cancer is a carcinoma, lymphoma, blastoma, or sarcoma.

59. The method according to claim 58, wherein the sarcoma is a soft-tissue sarcoma (STS).

60. The method according to claim 59, wherein the STS is alveolar soft part sarcoma (ASPS).

61. The method according to claim 56, wherein the cancer is renal cell carcinoma (RCC), breast cancer, or melanoma.

62. The method according to claim 61, wherein the RCC is translocation RCC (tRCC) or TSC/mTOR-altered RCC, or wherein the breast cancer is GPNMB+ triple-negative breast cancer (TNBC).

63. The method according to claim 56, wherein cancer cells of the cancer are GPNMB negative, and wherein the cancer comprises a tumor microenvironment comprising GPNMB positive cells.

64. The method according to claim 55, wherein the condition associated with GPNMB expression and/or activity is a neurodegenerative disease.
